(19) United States
(12) Patent Application Publication
(10) Pub. No.: US 2006/0094645 A1
(43) Pub. Date: May 4, 2006

(54) Method for defining and treating chemically-induced immune disorders using tumor necrosis factor (TFNalpha), interleukin-1 (lL-1), and interleulin-6R(lL-6R) antagonists

(76) Inventor: Oliver Lawless, Highland, MD (US)

Correspondence Address: CONNOLLY BOVE LODGE & HUTZ LLP, SUITE 800, 1990 M STREET NW, WASHINGTON, DC, 20036-3425, US

(21) Appl. No.: 11/244,141

(22) Filed: Oct. 6, 2005

Related U.S. Application Data
(60) Provisional application No. 60/615,967, filed on Oct. 6, 2004.

(51) Int. Cl. A61K 38/17; A61K 38/13; A61K 31/573; A61K 31/525

(52) U.S. Cl. 514/11; 514/171; 514/251; 514/12

(57) ABSTRACT

Compositions and methods are disclosed for treating chemically-induced immune disorders. Pharmacological compositions comprising at least one TNF antagonist, at least one IL-1 antagonist, and at least one IL6R antagonist are disclosed.

### PRIORITY

This application claims the benefit under 35 U.S.C. § 119(e) of provisional application 60/615,967, filed Oct. 6, 2004, the entire contents of which is specifically incorporated by reference for all purposes.

### FIELD OF THE INVENTION

The present invention relates generally to compositions and methods for treating chemically-induced immune disorders. Pharmacological compositions comprising at least one TNF antagonist, at least one IL-I antagonist, and at least one IL6R antagonist are disclosed.

### BACKGROUND

The information provided below is not admitted to be prior art to the present invention, but is provided solely to assist the understanding of the reader.

Environmental Context. The advent of the modern era has brought innumerable advances in technology and the development of synthetic materials, changing the landscape of the environment with which humans must continuously interact. This ever-increasing artificial landscape has forced the transition from a symbiotic relationship between humans and the natural environment, to one in which humans must now contend with their immediate, man made, surroundings. Nowhere is this more evident than within the human body—where the human organism attempts to defend itself from traditionally natural antagonists as well as a plethora of new man-made or unnaturally occurring chemicals and compounds. Where traditional science and immunology once focused merely on protein/peptide triggers from potential pathogens and living organisms, a changing environment has revealed the need to shift immunology's theoretical foundations, by expanding a basic paradigm to incorporate both the introduction of inert triggers, and a new causal mechanism for immune responses and diseases. The CIIDs paradigm explains the impact of a new and changing causative environment, on immune function, from exposures, onset, diagnosis, to treatments, and eventually prevention. This paradigm helps to explain that while infectious diseases are declining in developed countries the prevalence of autoimmnue diseases such as Asthma, Diabetes and Multiple Sclerosis are increasing greatly (Bach N E J Med 2002). This has been ascribed to the Hygiene theory which implies that the absence of exposures to bacteria (good hygiene ) or early treatments with antibiotics of infectious organisms, prevents the population from developing protective immunity, and therefore causes autoimmune diseases (AID). (Weiss N E J Med 2002). This suggests paradoxically that infectious diseases protect one against AID, which is in direct contrast with the concept proposed that they cause AID by molecular mimicry (see below). Epidemiologically these AID are more prevalent in Northern rather than Southern climates, are related to per capita income (the higher the income the greater the prevalence). Confounding factors that have not been addressed in these studies are sunshine exposures, vitamin D analogues, vaccinations, chemical additives to foods and agricultural products, use of pesticides and herbicides, sophisticated medical drugs and devices afforded by a high income generating and consuming public. That the cause for the increase in AID in developed countries is environmental rather than genetic, is based on the findings that Pakistanis who emigrate to the UK have a 10 fold increase in Diabetes despite presumed similar genetics, and blacks in Africa have a much lower incidence of Lupus when compared with American blacks. (Bach N E J Med 2002) Recent data in the US cites a seventy percent increase in mortality in blacks from Lupus in the last 10 years (CDC 2002), and Arthritis in the US is at an all time high affecting approximately 70 million, with a prevalence per state varying from a low of 18 to a high of 41%. (CDC 2002).

Immune Tenets. Several elementary yet important immune tenets merit reflection here. They are often taken for granted due to their simplicity, but the complex bio-mechanics of the underlying organism have evolved to support these basic tenets. In the broadest sense, the primary goal of the mammalian immune system is to preserve homeostasis within the organism. The natural state of the system is flux, a feedback continuum, with continual destruction and regeneration at the cellular level. A result of this defining state is that every action or stimulus must be able to obtain a reaction or response, to continue trending toward homeostasis. The system's composition, as well as the resources it has at its disposable for defense may also be deemed “natural”: capable of being organically destroyed and regenerated. The organism's composition and adaptability, as well as that of its constituent parts, is physically limited only by its genetically coded building blocks (DNA and proteins) and capabilities, which it has attained over the course of its evolution. The system is self-contained and inter-dependent, and when posed with a threat, focuses on containment, since deviations in one area may affect the function of other areas. Thus the immune system, as well as its individual components, such as cells, organs, and tissues, operate with the functional goal-oriented aim of trending back to homeostasis. The mammalian immune system, an evolutionary marvel, is a continuum, limited only by the evolutionary tenets which it is based on. The significance of these elementary tenets is that there are degrees wherein violation does not result in organ or organism death, but will serve to severely disrupt cells, organs, and impair immune function.

Conventional Immune Theory. Historically, the innate/non-specific and adaptive/specific immune systems have been treated as separate functional entities, with independent roles and resources. Investigation of the innate system has been largely neglected due its limited complexity, resources, and role in an immunogenic response. However, regardless of the simplicity and limitations of its cellular components, it maintains a paramount role in communication between the two cellular systems and the coordination of an efficient adaptive immune response. (Janeway 1992).

The innate/non-specific immune system is the evolutionary older system, and is found in both invertebrates and vertebrates. In a simplistic sense, the innate immune system provides an initial non-antigen-specific response to a foreign substance, which upon subsequent encounter with the same substance, merely repeats the same general response (Bellanti, 49). Its primary cellular components are macrophages, dendritic cells (“DCs”), natural killer cells (“NKs”), and T cells (Arend, 2001). The functional integrity of these component cells enables rapid response kinetics. Macrophages and DCs recognize repetitive molecular patterns, or pathogen-associated molecular patterns (“PAMPs”) on their pattern recognition receptors (“PRRs”). All of their PRRs have the same specificities (Arend, 2001). The receptors which initiate these responses are called Toll Like Receptors and now number at least 11, (Aderem 2000) and are increasing in both number and specificity almost daily. As a result, phagocytes and DCs have a limited number of pre-existing cell types and responses. The adaptive/specific immune system, found only in vertebrates, is more discriminatory, and may be characteristically distinguished from the innate system through its specificity, heterogeneity, and memory upon subsequent antigenic encounters (Bellanti, 49). All three adjectives describe the increasingly complex roles played by lymphocytes such as T cells and B cells, which can selectively proliferate and differentiate themselves upon subsequent exposures to antigens.

Macrophage play a central role at the innate/non-specific level. At various times, they serve dual, but fundamentally differing functions. Basal state macrophage function is centered on homeostatic functions such as the phagocytosis and elimination of proteins and apoptotic cells and debris. However, it also functions as the first line of immunological defense and becomes activated in response to external threats. Much of the destructive capacity of the immune system, with respect to tissue damage, is invested in macrophages. The activated phenotype is histotoxic, with a potent ability to initiate adaptive immunity and capacity to kill cells, organisms, or antigen. As a consequence, the role of the activated macrophage under “normal” conditions is limited in terms of specificity and duration. The critical difference in its functional efficacy is its ability to initiate inflammation. During receptor activation, and cellular signalling a process of altered gene expression, coordinated by contact with cytokines, extracellular matrices, cell-bound ligands, and microbial products leads to temporary production of the proinflammatory cytokines TNFα, IL-1, IL-6, IFNα, and IFNγ_(Nathan and Muller, 2001 through activation of its Toll like receptors. (Rehli 2002). Activated macrophage produce macrophage derived interleukins (“MDI”) in staggered intervals over a four hour time cycle which serve to degrade the causative agent, alter chemokine migratory patterns, chemo-attract, activate and differentiate cells associated with the response. The macrophage's response to the two forms of cell death has been shown to be rather important, since the induction of inflammation brings with it numerous secondary consequences. It has been shown that cellular apoptosis does not initiate a macrophage-based inflammatory response, and in fact may be inflammatory suppressive (Cocco and Ucker, 2001). Cellular apoptosis is characterized by an orderly sequence of internal events. Chromatin condensation precedes the loss of cellular integrity, thus providing a means of containment for nuclear components (Russell, 1983; Wyllie et al, 1984; Harvey et al, 2000). As a consequence of this containment, bystander cells remain unaffected by intracellular processes (Ucker et al, 1989; Dhein et al, 1995). In contrast, necrotic cell death is characterized by rapid, disorganized swelling and rupture and is associated with pathological tissue injury (Henson and Johnson, 1987). The phagocytic response to necrotic or inflammatory cell death and foreign threats is marked by the initiation of inflammation (Henson and Johnson, 1987; Stern et al, 1996) for containment and elimination by itself and other cells attracted to the site by chemotaxis. It is now generally recognized that the inappropriate production of TNFα, IL-1 and IL-6 leads to continuous inflammation, tissue destruction and organ injury (Beutler and Cerami, 1986; Edwards et al, 1994; Ksontini et al, 1998). As such, an active program of cytokine mediated suppression is initiated upon containment and elimination of the causative agent. This suppression phase reorients the cell toward trophic functions such as wound healing (Nathan and Muller, 2001). However, perceived macrophage failure such as the inability to kill or contain the causative agent will escalate the level and specificity of the immune response. Accordingly, the immune system transitions its defense from its non-specific to its specific mechanism of defense. The cellular protagonists which drive the specific arm of the immune defense are T cells and B cells, and the macrophage's role after this transition is largely ignored.

The adaptive/specific immune response is defined by the presence of clonally expanded antigen-specific T and/or B cells in response to a specific antigen. The 2-signal model is the currently accepted model for specific immune activation. It posits that specific immune induction requires 2 signals (Bretscher and Cohn, 1970). The cause of induction of the initiating signal is still under debate. Signal 1 itself is an interaction involving antigenic protein/peptide transfer between the antigen-presenting cell (“APC”) and either the T cell receptor (“TCR”) on naive T cells or cell-membrane-bound immunoglobulins on B cells (cite). Activated macrophages or dendritic cells (“DCs”) may function as APCs, although it has been shown that macrophages are weak APCs in comparison with mature DCs. Contact with proinflammatory cytokines, microbial products, and antigens induces DC maturation, characterized by expression of class I and II major histocompatibility complex (“MHC”) molecules and costimulatory molecules such as CD80 and CD86 (Arend, 2001). These mature DCs present antigen to CD4+T cells via class II MHC complexes, to CD8+T cells via class I MHC complexes, and potentially endogenous and exogenous lipids and glycolipids via molecules of the CD1 family (Arend, 2001). Signal 2 consists of costimulatory molecules such as the proinflammatory cytokines TNFα, IL-1, and IL-6, CD2/CD58, CD40/CD40 ligand, CD28/B7, B7.1, B7.2, B7.3, CD58/LFA3, and or adhesion molecules which assist in priming the CD4+/T helper cells (Davis and van der Merwe, 1996). When both signals are present, T cells clonally expand and differentiate based on the milieu of surrounding cytokines. A theory espoused by Matzinger is that danger in the form of tissue damage or cell stress triggers the response termed signal 2 (Galluci and Matzinger, 2001). The nature of the Danger signal has not yet been proven but heat shock proteins (HSP )have been suggested (Corrigall 2002), and also found to be contaminated with endotoxin ( ) a known and potent innate activator. This APC/DC T cell interaction is now termed the Immunological Synapse. (Grakoui 1999).

A Th 1 response, involved in cell-mediated immunity and some auto-immune diseases, is manifested by delayed type hypersensitivity to the inducing antigen and produces the cytokines IL-2 and IFNγ (Mossman and Coffman, 1989). A Th 2 response, involved in humoral immunity, some auto-immune and allergic diseases, results in the production of IL-4, IL-5, IL-6, IL-10, and IL-12 (Mossman and Coffman, 1989). Failure to eliminate the antigen at the Th 1 lymphocyte cell level elicits Th 2 T cell activity, which in turn initiates B cell function (Abbas, 1994). B cell activation produces the antibodies IgG, IgM, IgA, IgD, and IgE, depending on the anatomical location of the antigen being presented.

With time and antigen persistence, an initial IgM antibody switches to an IgG antibody and the avidity of this antibody increases progressively with the aim of complexing, precipitating, and localizing it for phagocytic removal. On occasion, a secondary antibody may be formed against this antigen-antibody complex for the same purpose. A new IgM antibody directed against an IgG antibody complexed to antigen is called a Rheumatoid Factor (RF). It is therefore an “auto-antibody”. This RF used to be considered specific for Rheumatoid Arthritis and is indeed one of the criteria for its diagnosis. While characteristic this disease it can also be found following persistent antigenic stimulation by pathogens (Syphilis, Leprosy, Bacterial Endocarditis), and by chemicals (silica in silicosis, and mercury in mercury toxicity), and in other autoimmune diseases. (Mandell 1990) The time cycles of each of these activated lymphocytes is specific and measurable. The T cell response is measurable at 3-7 days, while B cell IgM antibody production peaks at 10 days. There is thus a systematic and ultimately measurable response progression by the different cellular arms which comprise the total immune response.

One of the potential byproducts of any persistent inflammatory immune response, repeated vaccinations, persistent infectious, and aging, is an autoimmune reaction. Auto-immunity is a condition known to arise when T cell function and B cell-derived antibodies are initiated against self-antigens, organic self-proteins and self-peptide structures. There is an important distinction between an autoimmune reaction and an autoimmune disease, however. Autoimmune reactions, according to Matzinger, are a normal part of immune responses. They are greater early in a primary response than in a late primary or secondary response because the autoreactive cells will eventually become tolerized by the presence of the autoantigen, in the absence of second signals (Matzinger, 1994). Autoimmune reactions are typically ephemeral. Under normal conditions, a mechanism known as tolerance functions to delete or prevent these responses from continuing unabated. A tolerized state may be defined as the absence of specific T cell and/or B cell antibody responses to self antigens in normal individuals. It has been proposed that tolerance may occur both centrally and peripherally, and the tolerance process may involve more than one mechanism. In central tolerance, T cell precursors migrate from the bone marrow to the thymus, where variable regions on their TCR genes are assembled by somatic DNA rearrangement (Kretz-Rommel and Rubin, 2000). During this process, only maturing thymocytes with TCRs which can positively interact with self-peptides presented by the MHCs on cortical thymic epithelial cells may proceed to development (Kretz-Rommel and Rubin, 2000). T cells with a high affinity for self-peptides will be negatively selected, or killed, and thus impeded from proceeding as auto-reactive cells into the periphery. Their non-responsiveness to self-antigens is acquired by increasing their activation threshold to self-peptides during positive selectivity in the thymus.

A number of passive mechanisms have been proposed to account for peripheral tolerance including induction of T cell anergy, T cell deletion, and immunological ignorance (lack of exposure of tissues or cells to lymphoid cells ), as they exist at immune privileged sites-the eye, and the brain, are examples. Active mechanisms that have been proposed include suppressor or regulatory T (TR) cells, including CD4+CD25+ TR cells. Important determinants in the differentiation of naive CD4+T cells are the APCs that stimulate them and their corresponding cytokine environment. It has been shown that IL-10 functions as a differentiating factor for TR cells. IL-10 also prevents DC maturation, which may assist in TR development, as T cells which are stimulated with immature DCs lead to differentiation into regulatory T cells (Maloy and Powrie, 2001). There is currently no way to delineate an exact molecular pathway for the suppression mechanism, but Maloy and Powrie have proposed a quantitative model for TR cell induced tolerance based on the ratio of TR-cells to TPATH cells (potentially pathogenic autoreactive T cells) which respond to a peripheral antigen (Maloy and Powrie, 2001). They assert that in the lymph nodes, this ratio is dynamic and fluctuates depending on the affected/inflammatory status of local tissues. During times of homeostasis, immature DCs (“iDCs”) circulate through peripheral tissues where they can phagocytise debris arising from natural cell turnover. In the absence of inflammation, some of these iDCs will migrate to the lymph nodes where presentation of self-antigens to both TR and TPATH cells is possible. Maloy and Powrie argue that an autoimmune reaction will not be initiated because the TPATH cells are insufficiently activated by the iDCs in the absence of signal 2 and/or the iDCs preferentially stimulate TR cells, since TR cells can respond more efficiently to low levels of cognate peptide ligands than conventional naive CD4+T cells. However, the onset of an inflammatory response to a causative agent will induce DC activation and subsequent migration to the lymph nodes for presentation of antigen/pathogen protein/peptide sequences, as well as self-antigen sequences resulting from inflammatory-based tissue destruction.

This DC stimulus (signal 1) combined with signal 2 will temporarily override TR cell activity and functionality, since the TR subset will also undergo cell division and clonally expand instead of actively suppressing the antigen/pathogen being presented. As the causative agent is cleared from the system and presentation of peptides by activated DCs diminishes, any effector T cells used in the combatative response will either die or become memory cells. It is now known that TNFα plays a dual role here in that it both initiates and terminates specific immune T cell involvement, the latter by T cell apoptosis by a process known as activated immune cell death AICD (Kokias 2002). Without costimulation, immature DCs will again process cellular debris in a non-activated state. Since both subsets of T cells underwent proliferation, the initial ratio may be maintained, such that TR cell regulatory activity may predominate, inhibiting TPATH cells and down-regulating APCs (Maloy and Powrie, 2001). They also note that these TR cells may migrate into the periphery and thus perform a similar function.

Recently “Innate Tolerance” induced by endotoxin has been suggested as one mechanism for down regulating MDI production thereby reducing atopy, allergies and asthma in children living on a farm and exposed to it, endotoxin, and milk proteins. (Braun-Fahrlander 2002). Matzinger's rules of tolerance in relation to her Danger Theory postulate that thymocytes and virgin T cells should die if they receive signal 1 in the absence of signal 2, and virgin T cells may only respond to a signal 2 offered by an APC. Effector T cells should die if they receive signal 1 in the absence of signal 2, but may receive signal 2 from a variety of different cell types. B cells should also die with receipt of signal 1 in the absence of signal 2, and should only receive this signal 2 from effector T cells. Lastly, effector cells should ignore signal 2, perform their predetermined function with the receipt of signal 1, and return to a resting state in a short time frame (Matzinger, 1994).

In all cases, the onset of auto-immune disease is considered to be the result of a disturbance in tolerance or error in some aspect of the immune response. Free radicals, cytokines, oxidative reductive enzymes, reactive oxygen species (ROS), and nitrogen species (NO), prostaglandins, heat shock proteins, superantigens, and polyclonal T and B cell activators have all been suggested, but none proven (Matzinger, 1998). Examples of self-antigen links to auto-immune disease include T cell responses and B cell responses with the production of antibodies to nucleosomes, splicing ribonucleoproteins, DNA, RNA, and Sm antigens in Systemic Lupus Erythmatosus (Casciola-Rosen et al, 1994), antibodies to alpha Topoisomerase 1 and to centromere antigens in Scleroderma (Casciola-Rosen, 1997), RNP antigens in Mixed Connective Tissue Disease Sharp, 1977), aminoacyl tRNA in Myositis (Tan,1992), antibodies to thyroid microsomal peroxidase and thyroglobulin antigens in Thyroid Disorders (Rose and McKay, 1985), and islet cell insulin and GAD65 antibodies in Diabetes Mellitus (Wucherpfennig, 2001) and to IgG as Rheumatoid Factors in Rheumatoid Arthritis. In these examples, it is considered that these specific T cell and antibody/B cell responses are in fact the causes of these diseases.

Viral, bacterial, and other infections and/or their fragments have been thought to cause autoimmune disease. The two theoretical mechanisms by which infectious agents might initiate autoimmunity are currently classified as either “antigen-specific” or “antigen-nonspecific”. The principle explanation for antigen-specific route to autoimmunity is epitope mimicry. The theory asserts that antigenic determinants on the proteins of a foreign microbe are similar to that of a host protein, yet different enough to be recognized as foreign by the host immune system (Benoist and Mathis, 2001). An example here is a streptococcal infection which is followed by a systemic illness potentially affecting the joints—arthritis, the heart—carditis, and the brain—chorea. For T-cells, those determinants would be linear peptide stretches about 8-15 amino acids long. Credibility for an imperfect determinant copy has been enhanced by the fact that T-cell receptor (“TCR”) recognition of MHC-peptide complexes is degenerate, and doesn't require primary structure homology between two peptides presented by a given MHC molecule (Wucherpfennig, 2001; Maverakis et al, 2001). A specific immune response to the microbe could then cross-react with the overly similar host tissue, facilitating a self-antigen specific response and potential autoimmunity (Benoist and Mathis, 2001). This hypothesis is supported by the fact that pathogen-specific T cell clones cross-react with specific self-peptides and cause disease in animal models. Further support for the molecular mimicry theory comes from the fact that B-cell tolerance can be broken when self-epitopes are displayed in a repetitive manner on viral surfaces (Bachmann and Kopf, 2001).

The most popular antigen-nonspecific explanation for pathogen induced autoimmunity is the bystander T cell phenomenon. Infections and other foreign pathogens result in cellular damage, either through pathogen-derived cell death or the action of cytotoxic immune cells and molecules. Self-antigens are a byproduct of necrotic cell death and may be absorbed and processed by macrophages and DCs. These APCs are then indirectly activated by non-specific factors Signal 2, or Danger, resulting in upregulation of costimulatory molecules and APC migration to secondary lymphoid organs where they may prime T cells to respond (Bachmann and Kopf, 2001). This supports the argument that indirect activation of APCs may trigger self-antigen lymphocyte responses and autoimmunity. It is compatible with the findings that administration of LPS together with thyroid extracts are able to overcome tolerance and trigger autoimmune thyroiditis (Weigle, 1980), and with the concept that metallic chemicals Gold, Mercury, Silica cause autoantibodies and autoimmune disease. Another and increasingly pertinent consideration is Cytokine Inbalance, where autoimmune T cells fail to be eliminated, either due to persistent trigger or ineffective activated immune cell death (AICD).(Kokias 2002) failure of-upregulation of IL-19 and TGFO both MDI regulators, innate tolerance induction, or regulator T cells.

Chemically Induced Immune Disorders (CIID). It is at this point which we address the issue of antigen composition, the starting point for any immune response. The CIID paradigm asserts that antigen composition serves as the trigger which creates a dysfunction in both arms of the immune system, ultimately leading to the onset of a diverse variety of diseases and disorders. The model may be delineated as a simple causal chain, commencing with exposure to triggers of a specific composition.

Antigen Composition. There are an increasing number of “inert” chemicals which, upon entry into the mammalian organism, become inert “antigens”, or CIID triggers. The inert nature of these triggers, or super adjuvants, is their defining characteristic. They may be inert because either they are synthetic in composition, because they should be present only as trace elements intra-cellularly, or because technology has enabled exposure to an element or chemical which otherwise would not be presentable to the mammalian organism. As a consequence, these triggers are impervious to “natural” metabolic processes; they may not be broken down via the cellular processes of any evolutionary-based organism. Since it is difficult for an organism to alter the structural composition of a CIID trigger once internalized, there is an implicit lack of feedback for the system to work with; any action taken to remove or degrade these triggers will not necessarily evoke a reaction or response from the trigger. Within an organic system, this type of feedback is critical to the recognition of success or failure of a metabolic process, as well as the maintenance of a homeostatic continuum. To frame it in an evolutionary context, the mammalian organism and its immune defense mechanism are the product of evolution. As such, the system's state of flux and the organism's pliability are evolutionary prerequisites. Violations of these basic tenets create evolutionary and systemic impediments.

We define a CIID trigger to be any inert substance such as a synthetic material, heavy metal, chemical, or crystal not genetically programmed to exist independently at the cellular level of the mammalian organism. Examples include silica, silicate, silicone, beryllium, titanium, platinum, lead, mercury, iridium, zinc, manganese, iron, cobalt, gold, silver, cerium, vanadium, tungsten, cadmium, gold, silver, bismuth, aluminum, iron, tantalum, germanium, thorium, and certain of their salts and other rare earth metals, crystals such as uric acid, calcium pyrophosphate, and hydroxy-apatite, persistent organic pollutants and their metabolites such as DDT, DDE, endrin, hexachlorobenzene, heptachlor epoxide, trans-nonachloroxychlordane, and lindane, non-persistent organic pesticides and their metabolites such as atrazine, diazinon, malathion, chlorpyrifos, and pyrethroids, polyaromatic hydocarbon metabolites such as hydroxybenzoapyrene, hydroxybenzoepyrene, hydroxyfluoranthene, hydroxyphenanthrene, hydroxychrysene, and hydroxypyrene, phthalate metabolites such as monoethyl phthalate, monobutyl phthalate, monoethylhexyl phthalate, and monoocytl phthalate, each of which is detectable in the blood or tissues of the afflicted individual.

Medical implants such as breast, chin, testis, penile, lip, pacemakers, defibrillators, nerve stimulators, insulin and morphine pumps, shunts, catheters, siliconized coils-dialysis heart lung by pass tubing—and others, are all comprised of some combination of the aforementioned chemicals and thus serve as CIID triggers. Some of these elements may constitute part of the overall genetic constitution of the organism as trace elements, but are contained within the cellular framework on which the organism is based. The distinction lies in the fact that the element may be a component of a cell and functional cellular network, but should not occur externally to the cell, as an independent free floating molecule. For example, aluminum should be present in XX quantities in XX types of cells, but should not be present as an isolated molecule.

Methods of Exposure. Methods of exposure to any of the CIIDs triggers may include inhalation, ingestion, including dental manipulations, skin absorption, and by the subcutaneous, submuscular, intra muscular, intra-organ, intra venous, intra arterial, infusions, injections and delivery of vaccines, chemicals, drugs and devices. The magnitude of the response and number and type of resulting CIIDs are directly dependent on the stimulus created by the trigger, which in turn is directly dependent on the method, dose, duration, and frequency or permanency of exposure(s), as well as the genetically controlled susceptibility of the individual. The response range is therefore highly correlated with the dose, duration, size of particulate, chemical reactivity/noxiousness of the trigger's composition, site, degree of localization or containment, genetic susceptibility and capacity to produce certain enzymes capable of metabolizing certain xenobiotics, and anatomical location of the trigger's presence. Ultimately, time or duration of the trigger's presence and genes are the most important factors in the magnitude of the response.

The National Report on Human Exposure to Environmental Chemicals is a new study which assesses the exposure of the U.S. population to environmental chemicals using biomonitoring. Environmental chemicals were defined as chemical compounds or elements present in air, water, soil, dust, food, or other environmental media. Assessment of exposure was conducted by the measurement of 27 chemicals and their metabolites in the blood and urine samples of healthy human specimens. Chemicals sampled included 15 metals, the metabolites of 28 pesticides and 7 phthalates, and cotinine, a metabolite of nicotine. The metals cadmium, lead, mercury, antimony, barium, beryllium, cesium, cobalt, molybdenum, platinum, thallium, tungsten, and uranium were all sampled. All but two, beryllium and platinum were identified in the blood or urine at geometric mean levels between a low of 0.0008 ug/L for uranium and a high of 48.8 ug/L for molybdenum. All organophosphate pesticides were detected at levels of 0.51-2.61 ug/L, and four of the six phthalate metabolites were detected at 3.5-176.0 ug/L (CDC, March 2001). Each of the aforementioned chemicals and metabolites has no functional extracellular basis within the mammalian organism.

In a separate report on Trade Secrets aired by PBS (2001), Bill Moyers had his own blood tested for 150 chemicals, pesticides, and herbicides which should not naturally occur in the human body. The results returned positive identification of 84 of the 150 chemicals sampled, an astounding 56%.

In both of the above studies, the presence of various inert chemicals is statistically significant. Furthermore, it is critical to note that both studies were done on healthy populations not known to have any specific chemical exposure. Participants were screened to exclude forms of employment involving direct occupational chemical exposure such as smelting, coal mining etc. These findings when combined with the data showing major increases in AID and arthritis world wide which is linked to industrial wealth and per capita income, are provocatively suggesting positive correlation with CIIDs triggers, rather than a positive correlation with the absence of infections and the presence of over zealous “Hygiene”. The fourfold increased risk for Systemic Lupus Erythematosus in the Carolinas (Parks 2002) and other connective tissue diseases, following silica exposures is in keeping with this premise.

CIID Mechanism. The mammalian organism views these CIIDs triggers as external threats because, in the most simple sense, they should not constitute any part of the organism's extracellular matter. The substances are not an inherent part of the organism's pre-determined genetic make-up. The presence of an independent chemical, element, drug, vaccine, or device in an extracellular milieu having no innate constitutional or functional purpose, or specifically designed as an adjuvant (Aluminum) or preservative (Mercury), in vaccines, displaces homeostasis within the system. This is in agreement with reports of immune response activation by immunization with self-proteins or peptides in complete and incomplete Freunds adjuvant, in animals with myelin basic protein which develop allergic encephalomyelitis, which is similar to multiple sclerosis in man. Immunization with cancer antigens from, for example malignant melanoma, and followed by harvesting and reinjection of enhanced numbers of tumor infiltrating lymphocytes and IL-2 in melanoma patients, results in regression of their tumors, and destruction of melanin producing cells of the skin, Vitiligo an AID also. (Science 2002). Both examples are manifestations of sustained T cell effects to self antigens. From an evolutionary perspective, the organism has the potential to adapt within the framework of its genetic building blocks. However, from a composition perspective, these inert triggers lie outside the organism's universe. The CIID trigger creates a persistent presence which permanently disrupts homeostasis at the locus, if not extractable from the organism by natural means. As a consequence, the innate immune defense mechanism is activated, and tries to contain it by granuloma formation (Sarcoidosis, Wegeners Granulomatosis, Berylliosis), or formation of a capsule or large granuloma (capsule around a breast implant or pacemaker).

A macrophage-led response is initiated and the proinflammatory cytokines TNF_, IL-1, and IL-6 are produced to degrade the invader, deviate chemokine traffic into the affected area, and upregulate DCs and adaptive immune cells. It is at the non-specific level where the initial disruption in immune function occurs. The undegradable inert trigger leads to response failure at the non-specific level and fails to provide necessary feedback at the cellular level, since its presence is static. This lack of feedback may be interpreted mechanistically as the extractable protein or peptide sequence which is a prerequisite for APC-TCR interaction via class I or II MHC molecules and the activation of the adaptive immune mechanism is either absent or minimal.

Thus, the composition of the trigger creates a duration issue. The trigger may attain a state of permanence within the organism as it remains impermeable to phagocytic function while also impeding the activation of a specific immune response. In time, the macrophage reaches a stalemate wherein it can neither expel the antigen, extract a protein or peptide, turn itself off, or shut down proinflammatory cytokine production via a suppression mechanism. As a result, the macrophage's normally brief cytokine production cycle is repeated ad infinitum. We term this point crisis, where violation of basic tenets obstructs normal immune function. (T cell deficiency and Tuberculosis in Silicosis). The defining characteristic of this crisis state is that permanent homeostatic displacement created by the trigger sustains macrophage activation beyond evident failure. At some threshold point, the sustained release of macrophage-derived and other cytokines and inflammatory molecules, particularly TNFα, become detrimental at the cellular level. As the trigger proves immutable and the area becomes saturated with cytokines and chemokines, there is nothing to absorb or beneficially use the potentially noxious and adjuvant molecules. Although these MDI may retain their goal-oriented function, only a functional execution distinct from the original objective may occur. Cytokine function thus becomes unbalanced, and detrimental, damaging neighboring healthy cells and inducing cellular necrosis, as well as breaching containment, and circulating into neighboring joints and tissues, causing similar damage which self-perpetuates the cycle. An example here is Aluminum induced Macrophagic Fasciitis with Arthritis, in patients immunized with the Hepatitis B Vaccine which uses Aluminum as adjuvant, and also mercury as a preservative. The magnitude of this response is highly correlated with factors associated with the stimulus, including dose, location, duration, chemical composition, and genetic predisposition of the individual. The critical aspect of this bystander cell necrosis is the chaotic manner in which the cell disintegrates, leaving nuclear components exposed to an adjuvantized immune milieu.

Many studies have substantiated our hypothesis that the CIIDs mechanism is propagated via a lack of nuclear cell containment. For example, a group led by Bickerstaff found that transgenic mice with targeted deletion of the serum amyloid P component (“SAP”) gene developed antinuclear autoimmunity and severe glomerulonephritis, a phenotype resembling Systemic Lupus Erythmatosus (“SLE”), the prototypical autoimmune disease. SAP is a plasma protein which shows specific calcium-dependent binding to DNA and chromatin in physiological conditions (Bickerstaff et al, 1999). SAP binds in vivo to both apoptotic cells and nuclear debris released by necrotic cells. Other recent findings have also implicated that the auto-antigens targeted in SLE are clustered on the surface of apoptotic cells. A study by Scott et al found that APCs of mice lacking the Tyro-3 family of tyrosine kinases cannot clear apoptotic cells and consequently overproduce TNFα and develop a severe lymphoproliferative disorder accompanied by broad spectrum auto-immunity against nuclear components (Scott et al, 2001). Evidence by Mevorach et al mplicates failure of apoptotic cell clearance leads to TNFα overproduction and the initiation of the adaptive immune response (Mevorach et al, 1998). Finally, a study by Kondo et al found that cytokine-induced DCs had the ability to maintain T cell survival in vitro in absence of antigen, and result in modest proliferation in 4 of 20 experiments (Kondo, 2001).

It is precisely this bystander necrotic cell death and the continued stimulation from the enduring CIID trigger which sustains the proinflammatory cytokine cycle. Thus the system enters the second phase of its aberrant response. Phagocytic activity continues, but due to the nuclear breakup associated with bystander necrotic cell death, self-nuclear, nucleolar, protein and peptide fragments including DNA, RNA, Sm, centromeres, proteins, peptides, etc. become present in the extracellular matter where they are subsequently engulfed, processed, and presented by activated APCs to the specific T and B cells of the immune system in the presence of co-stimulatory molecules, adhesion molecules, MHC molecules, Danger molecules, and the interleukins TNFα, IL-1, and IL-6 acting as “Adjuvants.” As a result, various types of self-antigens in this new highly charged or adjuvantized milieu, result in autoantibodies created from the nuclear, and cytosol, break up of neighboring cells. The progression from aberrant response to disorder and ultimately disease is propagated through the sustained production of MDI from activated macrophages responding to the presence of an enduring CIIDs trigger and neighboring and systemic necrotic cells. As the response is perpetuated and autoantibodies proliferate, effective containment and suppression becomes ineffective, and unrealistic. Although tolerance mechanisms function to inhibit autoreactivity, the normal tolerant state is disturbed by elevated and sustained levels of TNFα, IL-1, IL-6, and other immunoactivators both locally and systemically. Active peripheral tolerance is overridden as sustained MDI production impedes the effectiveness of regulatory or suppressor T cells. With regard to the Matzinger tolerance hypothesis, the sustained presence of a self-antigen signal 1 and costimulatory signal 2 prevents specific immune deactivation and tolerance induction. The result is that suppressor tolerance effects are lost both locally and systemically. Several recent studies provide evidence supporting overridden tolerance in AID. Kretz-Rommel and Rubin recently reported that injection of PAHA, a procaine amide derivative used for treating cardiac arrhythmia, into the thymus of normal adult mice interferes with positive selection of thymocytes, leading to a break in B cell tolerance in the periphery. This new model of drug-induced lupus manifests many of the features of the classical autoimmune disease SLE, and contradicts the popular idea that autoimmunity only arises through defects in thymic negative selection (Kretz-Rommel and Rubin, 2001).

Thus, an adaptive/specific immune response is initiated against these self-antigens via the collection of T and B cells present in the periphery and the migration of activated APCs to the lymphoid organs. A recent finding by Ishikawa et al has shown that autoimmunity may be initiated via lymphocyte attraction to self-antigens in the periphery. Ishikawa et al induced lupus-like symptoms in transgenic mice by inducing overexpression of B lymphocyte chemokine (“BLC”) in the pancreas. The physiological role of BLC is to coordinate the production and maintenance of B cell follicles in the lymph nodes and spleen by attracting CXCR5 receptor expressing B cells. Upon stimulation by BLC, B cells produce lymphotoxin B, which is essential for the generation of follicular DCs, formation of B cell follicles, and production of secondary lymphoid chemokine (“SLC”), which attracts T cells and leads to T cell regions adjacent to the B cell follicles. Ishikawa et al found that enhanced expression of BLC in DCs of these transgenic mice may attract B1 cells and T cells, creating a lymphoid environment in a non-lymphoid organ—the pancreas, a situation suited to cause autoimmunity (Bachmann and Kopf, 2001). Thus, the initial deviation affects immune function in other areas of the organism and the initial CIIDs trigger may in fact be responsible for a number of different CIIDs, and secondary diseases. The increased frequency of other AID in specific autoimmune illnesses such as Lupus and Sjogrens, and the increased frequency of death from coronary artery disease, newly considered to be immune based, also, in these AIDs, is in keeping with this premise. In further emphasis of this latter finding is a reduced incidence of coronary and carotid artery disease in patients with a mutation on the, TL 4, toll-like receptor 4, which is resident on macrophages, endothelium and other cells, and which is activated by LPS and endotoxin as well as other chemicals, with the reduced production of MDIs compared to normals. The MDI response levels, in this mutated genotype, has therefore been considered as the reason for the reduced frequency of arteriosclerosis. In TOLL receptor knockout mice, arteriosclerosis is totally prevented, affirming this mechanism.

The type of self-antigens and autoantibodies created and consequently the resulting type of CIID varies depending on several factors. The nature, the type, and the duration of these disorders and diseases is dependent on the genetic susceptibility of the individual, which influences Toll-Like receptor activity, T cell receptor αβ and γδ, and subset, the type, titer, class, and persistence of the antibodies made, the levels and persistence of the macrophage-derived interleukins, and the genes, SNPs, messenger RNA, and activation and signalling factors responsible for their production. Evidence that genetic factors play some role in autoimmunity come from studies of familial disease clustering, concordance rates in monozygotic versus dizygotic twins, and studies of alleles in the histocompatibility locus antigen (“HLA”) cluster (Cooper at al, 1999). The consensus of most workers in the field is that these AIDs are dependent upon multple genes-polygenic, and that environment is playing an increasingly important role. If the immunogenic event occurs locally, the disease caused by immune activation (Innate, T cells, or antibodies,) will remain local in the organ affected, but it in turn will become increasingly destroyed by this innate, specific, and auto-immune process. Organs, and their cells, may have a predilection for localization of environmental and pharmaceutical chemicals. Examples are iodine and thyroid disease, arsenic in water and diabetes, (water contamination by arsenic has been associated with diabetes in both Taiwan and Bangladesh), elevated air pollutants and asthma, and lung disease, such as World Trade Center Illness (elevated PM10s have been associated with obstructive lung disease, asthma, and a mortality rate in major cities increased by 1% per day from cardiorespiratory illness when these PM10s are increased (over a threshold level.) Aluminum and silica, copper, iron and zinc are identified in the brains of Alzheimer's disease patients. The brain is considered an immune privileged site and its evolutionary development preceded the development of T and B cells, thus these cells are normally not present there. The response to antigens or triggers at this location is via the Innate system of glial cells which have a Phagocytic function. The deficiencies caused by single organ failures may have systemic effects and consequences, particularly if they take place in the endocrine glands or system, brain, kidney or heart, which have major controlling hormonal effects on all other organ and cellular systems throughout the body. If this event occurs systemically, as takes place in classical immune diseases, there are major systemic consequences for the organism. However, once initiated and uncontrolled, a cascade of other immune happenings ensue such as autoantigen spreading, antibody avidity enhancement, complement activation, and others.

Chemically Induced Immune Disorders. The specific type, and gravity of a Chemically Induced Immune Disorder, is the result of the magnitude and type of chemical stimulus, which is in turn determined by the dosage amount and frequency of the chemical exposure, the chemical reactivity, anatomical location of its presence, its persistence, the genetic predisposition of the individual to any particular disorder, and most importantly, duration of the exposure, and the capability of it's elimination. With time, the persistent presence of a CIIDs trigger may result in the manifestation of any of the following CIIDs:

Usual Occupational Lung Disorders including Silicosis, Asbestosis, and Berylliosis.

Silicone Implant Illness, also termed Siliconosis.

Fibromyalgia, Chronic Fatigue Syndrome, Postural Hypotension, and Non-Specific Arthritis.

Atypical Connective Tissue Diseases or Auto-Immune Diseases (termed Atypical if they fail to meet the defined American College of Rheumatology criteria, and are caused by a CIIDs trigger, and termed Auto-Immune only when they meet and fulfill the ACR criteria) including Systemic Lupus Erythematosus, Sjogren's Syndrome, Scleroderma, Polymyositis, and Mixed Connective Tissue Disease.

Atypical Vasculitis, Relapsing Polychondritis, Wegener's Granulomatosis, Sarcoidosis, and Systemic Vasculitis including Polyarteritis Nodosa, Bechet's Syndrome, Churg and Strauss Vasculitis.

Atypical and Cryptogenic Lung Disorders including External Inhalant Alveolitis and Interstitial Lung Fibrosis.

Unusual Chemically Induced Occupational Pneumoconioses identified in response to chemicals such as beryllium, titanium, platinum, lead, mercury, iridium, zinc, manganese, cobalt, gold, silver, cerium, vanadium, tungsten, cadmium, bismuth, aluminum, iron, and certain of their salts and other rare earth metals including tantalum, germanium, and thorium.

Atypical Skin Disorders including Non-Specific Dermatitis and Vasculitis.

Atypical Neurological Disorders including Parkinsonism, Non-Infectious Meningitis and Cerebritis, Cerebral Atrophy, Peripheral Neuropathy, Carpal Tunnel Syndrome, and Cognitive Dysfunction.

Neurodegenerative Brain Disorders including Alzheimer's disease and Unidentified Bright Objects UBOs on MRI brain scans.

Blood Disorders including Polyclonal Gammopathy, Monoclonal Gammopathy (benign and malignant), multiple Myeloma, Amyloidosis, and autoantibodies to gammaglobulin (Rheumatoid Factor), nuclear antigens DNA, RNA, histones, and nuclear enzymes myeloperoxidase, topoisomerase, and aminoacyl and alanyl synthetases, cardiolipids, collagen and glomerular basement membrane.

Atypical Kidney disorders of focal and diffuse and crescentic Glomerulonephritis, Nephrotic Syndrome, Interstitial Nephritis, and Goodpastures Syndrome.

Atypical Endocrine disorders including Diabetes Mellitus.

Atypical Thyroid disorders including both Hypo and Hyper Thyroidism.

Cancers of the bone—Multiple Myeloma; of the lung—Adenocarcinoma; of the pleura—Mesothelioma; and certain B cell Lymphomas and Leukemias.

Previously unidentified diseases such as Persian Gulf War Illness and Desert Storm Pneumonitis/El Askhan Disease and World Trade Center Illness.

There exists a need for treatments for chemically-induced immune disorders.

Other objects and advantages will become apparent from the following disclosure.

### SUMMARY OF INVENTION

An aspect of the present invention provides pharmacological compositions for treating chemically-induced immune disorders (CIID) in a mammal. According to an aspect, the pharmacological composition comprises at least one TNF antagonist, at least one IL-1 antagonist, and at least one IL6R antagonist.

An aspect of the present invention provides methods of treating chemically-induced immune disorders in a mammal. According to an aspect, the method comprises administering to a mammal a pharmacological composition comprising at least one TNF antagonist, at least one IL-1 antagonist, and at least one IL6R antagonist.

According to an aspect, the diseases, syndromes, and/or conditions treatable by the compositions and methods of the present invention include, but are not limited to: Silicosis, Asbestosis, Berylliosis, Silicone Implant Illness/Siliconosis, Chronic Dialysis Syndrome with Dementia Neuromyopathy and Nephrogenic Fibrosing Dermatopathy, Fibromyalgia, Chronic Fatigue Syndrome, Postural Hypotension, Non-Specific Arthritis, Systemic Lupus Erythematosus, Sjogren's Syndrome, Scleroderma, Polymyositis, Mixed Connective Tissue Disease, Atypical Vasculitis, Relapsing Polychondritis, Wegener's Granulomatosis, Sarcoidosis, Systemic Vasculitis including Polyarteritis Nodosa, Bechet's Syndrome, Churg and Strauss Vasculitis, Non-Specific Meningitis and Cerebritis, Cerebral Atrophy, Peripheral Neuropathy, Carpal Tunnel Syndrome, Major Cognitive Dysfunction, Alzheimer's Disease, Unidentified Bright Objects on MRIs, External Inhalant Alveolitis, Interstitial Lung Fibrosis, Unusual Occupational Chemical Pneumoconioses, Polyclonal Gammopathy, Monoclonal Gammopathy, Multiple Myeloma, Amyloidosis, Glomerulonephritis, Nephrotic Syndrome, Interstitial Nephritis, Goodpasture's Syndrome, Diabetes Mellitus, Hypo and Hyper Thyroidism, Multiple Myeloma of the bone, Adenocarcinoma, Mesothelioma, certain B cell Lymphomas and Leukemias, auto-antibodies to gammaglobulin and the nuclear antigens DNA, RNA, histones, nuclear enzymes such as myeloperoxidase, topoisomerase, aminoacyl and alanyl synthetases; cardiolipids, collagen and glomerular basement membrane, Persian Gulf War Illness, and Desert Storm Pneumonitis/El Askhan Disease, and the World Trade Center Illness, Non-Specific Dermatitis, Vasculitis, Parkinson's Disease, and Non-Infectious chronic inflammatory systemic disorders.

Still other aspects and advantages of the present invention will become readily apparent by those skilled in the art from the following detailed description, wherein it is shown and described preferred embodiments of the invention, simply by way of illustration of the best mode contemplated of carrying out the invention. As will be realized the invention is capable of other and different embodiments, and its several details are capable of modifications in various obvious respects, without departing from the invention. Accordingly, the description is to be regarded as illustrative in nature and not as restrictive.

### DETAILED DESCRIPTION OF A PREFERRED EMBODIMENT.

Our observations suggest that by (i) avoiding exposure, (ii) early recognition,(iii) removing implanted devices and identified chemicals and (iv) down-regulating the products of innate immune cell activation by using the interleukin antagonists to TNFα, IL-1, and/or IL-6, one can prevent the initiation of the autoimmune processes and the resultant disorders and diseases which arise from exposure to these chemicals. Moreover, the compositions and methods of the present invention permit the treatment and amelioration of the resulting diseases, syndromes, and conditions with significantly less toxicity than the current treatment methods which employ long term corticosteroids and other chemotherapeutic agents. Treatment with interleukin antagonists, according to the present invention, will also result in a reduction in symptoms from both autoimmunity and proinflammatory overproduction, a reduction in signs of disease, normalized MDI levels in blood and tissues, and restoration of T cell function. Early treatment of these diseases will be easier and more successful than when the illnesses are fully established or chronic, where disturbed healing and fibrosis is dominant. In their latter stage, secondary events take place which are less dependent upon the TNFα and IL-1 and IL-6 interleukins than in the early stages of these diseases and disorders. An example is Scleroderma, a generalized fibrosing condition where over production of TGFβ and dysregulation of Smads which cause collagen overproduction by fibroblasts, is considered responsible for later phases (Varga 2002) following induction by silica (Lawless et al 2001). Similarly, early explantation of irritant chemicals is preferred when feasible, and even then, does not guarantee complete abatement of symptoms.

TNF and IL-1 antagonists to be used in the treatment of CIIDs are designed in two general ways: (i) acute regimens, designed to achieve rapid blood levels and rapid action, where the TNFα and IL-1 blockade is desired for a period of hours to days and (ii) chronic regimens, where the TNFα and IL-1 blockade is desired for a period of days, weeks, months or years.

TNF antagonists suitable for purposes of the present invention include, but are not limited to, etanercept (ENBRELTM®, Immunex Corp) and infliximab (REMICADETM® Centocor Corp.).

IL-1 receptor antagonists suitable for purposes of the present invention include, but are not limited to, kineret (ANAKINRA® Amgen Corp.).

Anti IL-6 R antibodies suitable for purposes of the present invention include, but are not limited to, MRA, and D2EF Adalimubab (HUMIRA® Abbott Corp.).

According to an aspect of the present invention, corticosteroids including, but not limited to, methotrexate, leflunomide, cytoxan, immuran, and cyclosporin A may be administered concurrently with the TNFα and IL-1 antagonists for treatment of any of the CIIDs outlined. In addition, corticosteroids, methotrexate, leflunomide, cytoxan, immuran, and cyclosporin A all produce immune system modulation in the standard treatment of idiopathic immune, and auto-immune diseases. These drugs all have direct anti-inflammatory properties as well.

An embodiment of the present invention provides pharmacological compositions for treating chemically-induced immune disorders in a mammal comprising at least one TNF antagonist, at least one IL-1 antagonist, and at least one IL6R antagonist. Persons of skill in the art may formulate suitable pharmacological compositions given the present disclosure and the teachings of Remington's Pharmaceutical Sciences (Gennaro et al., eds., Remington: The Science and Practice of Pharmacy, 20^{th} Ed., Lippincott Williams & Wilkins, Baltimore, Md. (2000)) the disclosure of which is hereby incorporated by reference.

A further embodiment of the present invention provides pharmacological composition for treating chemically-induced immune disorders in a mammal, wherein the TNF antagonist may include, but is not limited to, etanercept, infliximab, and mixtures thereof. The IL-1 antagonist may include, but is not limited to kineret. The IL6R antagonist of the invention may include, but is not limited to MRA, and D2EF Adalimubab, and mixtures thereof.

In further embodiments the pharmaceutical compositions of the invention may include a corticosteroid. Preferred corticosteroids may include, but are limited to of methotrexate, leflunomide, cytoxan, immuran, cyclosporin A, and mixtures thereof.

Currently TNFα antagonists are primarily marketed for use in Rheumatoid Arthritis (“RA”). It has been found that the T cell immunity in patients with RA is significantly reduced. Its use has been expanded successfully to the treatment of Crohns disease, Ulcerative Colitis, Psoriasis, Ankylosing Spondylitis and is being studied in other disease. Chronic TNFα stimulation of T cells in vitro leads to a similar phenotype (Isomaki et al, 2001). It is interesting to note that treatment with TNF antagonists can restore T cell function, and normalize deficiency of Regulator T cells, thereby restoring homeostatic mechanisms. This finding is in line with our CIIDs hypothesis outlining chronic homeostatic displacement as a mechanism for sustained autoimmunity. In vitro studies have also found that TNFα regulates the expression of other proinflammatory cytokines, and thus may be the most effective target to block the CIID mechanism we have outlined. This expands use of anti TNFs. Additionally, the observation that anti-TNFα treatments in patients with prototypical CIIDs such as silicosis and siliconosis blocks the propagation of the immune response provides definitive evidence that the proinflammatory cytokines, in particular TNFα, and IL-1, and or IL-6 are signal 2 costimulatory molecules (Lawless, 2001). The case for immune activation via homeostatic displacement and macrophage activation is evidence that the danger signal triggering signal 2 is simple homeostatic displacement by some form of extracellular matter not programmed to exist independently at the cellular level. The currently available data on patient outcomes in several thousands, which have not shown statistically significant adverse effects when compared with controls using methotrexate is encouraging. The activation of latent Tuberculosis appears both real and not unexpected, and warrants long term surveillance as well as for Lymphomas and Cancers which although increased in the diseases for which anti TNFs currently are used bears further study and observation.

The use of TNF, IL-1, or IL-6 antagonists, with or without the use of corticosteroids, methotrexate, leflunomide, and/or cytoxan for the treatment of CIID has not been described. In particular, the art lacks disclosure of compositions and methods of polytherapy comprising TNF antagonists, IL-1 antagonists, and IL6 antagonists for the treatment of CIID. The term polytherapy is defined herein to mean compositions and methods comprising antagonists to more that one class of interleukin. In particular, polytherapy is defined herein to encompass compositions and methods comprising TNF antagonists, IL-1 antagonists, and IL6 antagonists.

Furthermore, the art lacks disclosure of compositions and methods of polytherapy comprising TNF antagonists, IL-1 antagonists, and IL6 antagonists for the treatment of CIID where the CIID is chosen from Silicosis, Asbestosis, Berylliosis, Silicone Implant Illness/Siliconosis, Fibromyalgia, Chronic Fatigue Syndrome, Postural Hypotension, Non-Specific Arthritis, and Atypical Systemic Lupus Erythematosus, Sjogren's Syndrome, Scleroderma, Polymyositis, Mixed Connective Tissue Disease, Atypical Vasculitis, Relapsing Polychondritis, Wegener's Granulomatosis, Sarcoidosis, Systemic Vasculitis including Polyarteritis Nodosa, Bechet's Syndrome, Churg and Strauss Vasculitis, Atypical Non-Specific Dermatitis, Parkinson's Disease, Non-Infectious Meningitis and Cerebritis, Cerebral Atrophy, Peripheral Neuropathy, Carpal Tunnel Syndrome, Cognitive Dysfunction, Alzheimer's Disease, Unidentified Bright Objects on MRIs, External Inhalant Alveolitis, Interstitial Lung Fibrosis, Unusual Occupational Chemical Pneumoconioses, Polyclonal Gammopathy, Monoclonal Gammopathy, Multiple Myeloma, Amyloidosis, Glomerulonephritis, Nephrotic Syndrome, Interstitial Nephritis, Goodpasture's Syndrome, Diabetes Mellitus, Hypo and Hyper Thyroidism, Multiple Myeloma of the bone, Adenocarcinoma, Mesothelioma, certain B cell Lymphomas and Leukemias, auto-antibodies to gammaglobulin and the nuclear antigens DNA, RNA, histones, nuclear enzymes such as myeloperoxidase, topoisomerase, aminoacyl and alanyl synthetases, cardiolipids, collagen and glomerular basement membrane, Persian Gulf War Illness, and Desert Storm Pneumonitis/El Askhan Disease and World Trade Center Illness.

In particular, the art lacks disclosure of compositions and methods of polytherapy comprising TNF antagonists, IL-1 antagonists, and IL6 antagonists for the treatment of CIID associated with exposure to either silica, silicate, silicone, beryllium, titanium, platinum, lead, mercury, iridium, zinc, manganese, iron, cobalt, gold, silver, cerium, vanadium, tungsten, cadmium, gold, silver, bismuth, aluminum, iron, tantalum, germanium, thorium, certain of their salts and other rare earth metals, crystals such as uric acid, calcium pyrophosphate, and hydroxy-apatite, persistent organic pollutants and their metabolites such as DDT, DDE, endrin, hexachlorobenzene, heptachlor epoxide, trans-nonachloroxychlordane, and lindane, non-persistent organic pesticides and their metabolites such as atrazine, diazinon, malathion, chlorpyrifos, and pyrethroids, polyaromatic hydocarbon metabolites such as hydroxybenzoapyrene, hydroxybenzoepyrene, hydroxyfluoranthene, hydroxyphenanthrene, hydroxychrysene, and hydroxypyrene, phthalate metabolites such as monoethyl phthalate, monobutyl phthalate, monoethylhexyl phthalate, and monoocytl phthalate, and implants such as breast, chin, testis, penile, lip, pacemakers, defibrillators, nerve stimulators, insulin and morphine pumps, shunts, and siliconized coils.

The use of TNF antagonists used in monotherapy, but not polytherapy has been reported in cases of Sarcoidosis, Wegeners Granulomatosis Behcets Syndrome and Chronic Uveitis.

Examples of CIIDS Illnesses.

Silicosis and Coal Workers Pneumoconiosis (“CWP”). Silicosis and CWP are occupational diseases caused by the inhalation of coal dust, silica, and silicate particles and estimated to affect 1.2 million exposed workers (Speizer, 2000) such as miners, coal, gold, and uranium tunnelers, sand blasters, stone polishers, stone masons, and even diamond polishers. This exposure is associated with 100,000 deaths annually (Speizer, 2000). CWP and silicosis can be present as an acute, subacute, or chronic illness, and may be associated with tuberculosis, kidney disease, connective tissue diseases, vasculitis, Wegener's Granulomatosis, lung fibrosis, granulomas, skin disorders of pemphigus and bullous pemphigoid, and lung cancer. Dose, duration of the exposure, and genetic susceptibility determine both the outcomes and the life expectancy associated with these occupational diseases, a perfect fit within the CIIDs framework. No current treatment exists other than removal from the source of exposure. The etiology of this illness is a prototypical example of the CIIDs mechanism. Exposure to the inert trigger, silica and coal dust particles, initiates macrophage activation, proinflammatory cytokine production, prostaglandin formation, inducible nitric oxide formation iNOS, hydrogen peroxide and peroxynitrite generation, Fas and Fas Ligand activation, and resultant macrophage-induced cellular necrosis, adaptive immune activation, autoantibody production, and disease onset, progression culminating in lung fibrosis, and lung failure, or secondary associated diseases of Tuberculosis and Lung cancer. The significant outcomes of debility, disability, and death from the illness are preventable, but up to now, untreatable. The use of TNFα, IL-1, and IL-6 antagonists for the treatment of this illness provides a unique new tool for a currently untreatable illness and its complications, which include lung cancer, lung and kidney failure.

Unusual Atypical Metal Induced Lung Disorders. Inhalation of metal dusts and fumes can induce a wide range of lung pathologies (Kelleher et al 2000) including airway disorders, parenchymal lung disease, and cancer. The parenchymal diseases of the lung include chemical pneumonias, granulomatous lung disease, giant cell interstitial pneumonitis, and interstitial pneumonias. Kelleher details the metallic exposures that have been identified in their causation including exposures to aluminum, beryllium, cadmium cobalt, copper iron, mercury, and nickel. Aluminum causes interstitial lung fibrosis, sarcoid-like granulomas and alveolar proteinosis. Because it is often linked to the mining of bauxite, silica exposure and silicate particles are often found in the lung biopsy specimens, in addition to aluminum/silicate particulates. The best studied cases immunologically are those associated with beryllium exposure where both T cell and macrophage activation has been identified.

All of these diseases should be considered operationally to have three phases in their development: an initiating phase, a proliferative phase, and a healing phase. The initiating phase is comprised of exposure and early symptoms, at which time cold-like symptoms and non-specific bronchitis may be presented. The proliferative phase involves immune cell proliferation leading to granuloma formation. The healing phase involves fibrosis collagen production and results in scarring. During the initial phase, anti-TNFα agents are likely to be effective, particularly when coupled with removal from exposure. The granuloma and fibrosis phase are less likely to be responsive to these agents as other cytokines other than MDIs are operational and are similar to these phases in the development of Scleroderma. Early diagnosis, early removal from exposures, and early use of anti-TNF agents are likely to have major benefits in these and other CIIDs where currently no known treatments are available.

Asbestosis. Asbestosis is an occupational lung disease which results from inhalation of asbestos fibers. Asbestos fibers are mined in nature and are impregnated with silica. It is estimated that 250,000 persons are exposed in primary processing, with 3 million persons exposed to secondary processing and the estimated risks for the development of asbestosis lung disease is between 10% and 50%, and between 1% and 10% for lung cancer (Speizer 2000). The fibers are divided into two groups: Serpentine and Amphibole. Chrysotile, also called white asbestos, is a member of the Serpentine group. Amosite and Crocidolite, most commonly used in buildings, are members of the Amphibole group and are known as brown and blue asbestos, respectively. This disease results from the lung's response to both the fiber and silica components. Lung fibrosis and nodulosis follows, and results frequently in calcified fibrous plaques on the outer borders of the lung coverings or pleura. It also frequently results in lung cancer and an unusual form of cancer called Mesothelioma. Ultimately, lung function is compromised and individuals die from lung failure or the associated cancers. Smoking appears to increase the frequency of associated lung cancer. Recently new man-made fibers have been found to cause similar illness (Flock Worker's Lung), thereby reinforcing the CIIDs paradigm and mechanism for the illness. The use of TNF, IL-1, and IL-6 antagonists for the treatment of this illness provides a unique new tool for a currently untreatable illness and its complications, including lung failure and lung cancer.

Berylliosis. Berylliosis is an occupational lung disease associated with occupational exposure to the element beryllium. Estimated worker exposures in the U.S. is around 800,000 persons (Kelleher et al, 2000). The greatest exposure to beryllium occurs in the workplace, where it is mined, processed, or converted into alloys and chemicals. Acute exposure is by inhalation where it can cause acute pneumonia. The pneumonia can be mild, moderate, or severe and even acutely cause death. Chronic exposure causes chronic beryllium disease, or Berylliosis, an inflammatory fibrosis of the lung, which is also associated with nodular non-cancerous lung infiltrates. Symptoms of the disease may be delayed from months to years, and include irritation of the mucous membranes, reduction in lung capacity, shortness of breath, loss of appetite, weight loss, malaise and even death. Avoidance of and removal from the exposure source may result in some improvement in the symptoms. There is no standard treatment or this illness at this time. The use of TNFα, IL-1, and IL-6 antagonists for the treatment of this illness provide a unique new tool for a currently untreatable illness and its complications, including lung cancer.

Silicone Implant Illness/Siliconosis. It has been estimated that between 1 in 6 and 1 in 10 members of the U.S. population have some foreign material implanted in their bodies. Between 1 and 2 million women in the United States have undergone breast implant augmentation surgery with silicone gel and saline breast implants (Gabriel et al, 1967; Angell, 1994; Sanchez-Guerrero et al, 1994). The number with chin, testis, penis, retinal band, fallopian tube clamp, heart pace maker, heart defibrillator, morphine pump, insulin pump, nerve stimulator and other implanted devices is estimated to be over 5 million. In addition to the presence of silicones, these implanted devices contain other chemicals including polysulphones, epoxies, platinum, titanium, iridium, and others. The symptoms associated with silicone implants can vary from none to a plethora of non-specific symptoms such as Fibromyalgia, Chronic Fatigue, Postural Hypotension, Atypical Rheumatoid Arthritis, other Atypical Connective Tissue Diseases, Atypical Skin Disorders, and Atypical Neurological Disorders (Lawless, 2001).

It has been discovered that silicones (polydimethyl siloxanes), silicas (silicon dioxide), and silicates (sodium and other salts) cause activation of peripheral blood mononuclear cells (PBMs) in vitro in both normal and Silicone implanted patients, with concordant release of TNFα, IL-1, and IL-6 into the supernatants, with levels equal to that of lipopolysaccharide (“LPS”) or endotoxin, a known and potent activator of PBMs (Lawless and Ojo-Amaize, 1995). It has been discovered that silicone breast implanted patients have high levels of IL-1 (25%) and IL-1ra (75%) in their blood, compared with controls (Ojo-Amaize et al, 1996). T cells are activated in vitro in response to silicone, silica, and silicate in silicone breast implanted patients and not in controls. The activated T cells are identified to be of the T4 (CD4+) subset, as depletion experiments showed abolition of T cell proliferation upon CD 4 removal (Ojo-Amaize et al, 1994). Blocking experiments using monoclonal antibodies to CD 3, CD 4, and DR (MHC) also blocked proliferation in vitro (Ojo-Amaize et al, 1994).

Antibodies to silicate of both IgM and IgG classes have also been reported (Shen et al, 1996). Nature's attempt to localize infectious and noxious stimuli is by the formation of granulomas at the site of inoculation. Silicone implants are surrounded in the body by a container capsule, or pseudo-capsule, which closely resembles the synovial membrane of a joint involved with rheumatoid arthritis (O'Hanlon et al, 1995 and 2000). These breast capsules contain a plethora of immune cells including T cells, B cells, Plasma cells, and fibroblasts. Silicone globules are evident within these immune cell deposits. Activated T cell markers are also seen as evidenced by the activation marker CD 45RO (O'Hanlon 2000). Whether these T cells can “escape” from these sites of activation and disseminate throughout the body has been addressed. It has been discovered that the T cells resident in the capsules of patients with breast implants (which were identified by T cell receptor beta chain oligoclonal banding), were also found in the circulating blood pool, at both breast capsular sites, and in muscle, and skin biopsies, at three different locations which were the sites of clinical inflammation and vasculitis (O'Hanlon et al, 2000).

Thus, silicone, silica, and silicates in patients with breast implants cause innate monocyte/macrophage activation in vivo, in situ (capsules), and in inflamed tissues distant from the site of initiation or immune induction, causing an atypical Rheumatoid Arthritis with Vasculitis. It has been discovered that these findings of activated innate and adaptive immune responses (T and B) are not unique or restricted to silicone breast implants. They also have been identified in Saline Breast Implants, (the outer capsule is made of silicone), Retinal Bands (applied for retinal detachments), Fallopian Rings (for sterilization), heart pacemakers and defibrillators (Ojo-Amaize et al, 1995), and Silastic Joint Implant patients (Lawless et al, 2001). The findings of Chronic Fatigue, Benign Joint Hypermobility, Postural Hypotension, Raynaud's Phenomenon, Polmyalgia, Fibromyalgia, Peripheral Neuropathy, positive ANA (test for Lupus) and Polyarthritis were all found in a significantly increased frequency when compared with a normal control population. An increased frequency of thyroid gland abnormalities and diabetes which are classified as AID, were also unexpectedly, but not surprisingly found (Lawless and Ojo-Amaize, 2001).

There is a large literature of single and multiple case reports of Rheumatoid Arthritis, Systemic Lupus Erythematosus (Cooper et al, 1999), Scleroderma (Spiera et al) Mixed Connective Tissue Disease (Lawless, 2001), Sjogren's Syndrome (Bridges et al, 1993), Vasculitis (O'Hanlon, 2000), and Polymyositis (Love et al, ) associated with Silicone Breast Implants, and with silica exposures (Baur et al, 1996, Rodman, 1967, Steenland and Goldsmith, 1995, Steenland Brown, 1995, Tervaert et al, 1998, Parks, 1999, D'Cruz, 2000).Recently a case control study (Parks et al. 2002) in the Carolinas identified a 4.6 times increased risk for the development of SLE in those exposed occupationally or in farming to crystaline silica inhalation.

The dose of silicone implanted does not appear to predict or dominate the clinical or immunological responses, as up to 500 mgs of silicone gel is found in breast implants, and less than 10 mg in retinal bands and fallopian clamps, yet symptoms may be equally present in both (Ojo-Amaize, 1995; Lawless and Ojo-Amaize, 2001). The duration of the implants may play a role. We have discovered equal degrees of silicone specific T cell proliferation in individuals with retinal bands and breast implants, when the duration of implants is factored in (Ojo-Amaize, 1995; Lawless and Ojo-Amaize, 2001). The frequency of T cell proliferation appears to be important when assessed and correlated with the severity of illness criteria. Those with more severe illness were discovered to have a higher frequency of silicone specific T cell proliferation responses in vitro (Lawless et al, 1995).

In an attempt to discover the relative importance of innate and T and B cell responses in the causation of disease, we studied a patient with Sclero-Dermatomyositis (a classical autoimune disease), so termed because the overlapping symptoms of both these diseases were present in this patient. Both have been previously associated with silicone gel breast implants. This patient was an 85 year old (the disease is predominantly found in 3rd and 4th decades), male (both illness are more frequent in females) who had a heart defibrillator implanted ten months previously. Both findings caused us to question and identify: a) the ingredients in the defibrillator, b) the innate cell and T and B cell responses to the silicone antigens c) his genetic profile, and d) the auto-antibodies present. The pacemaker was manufactured by Medtronic and contained 4 silicones, 2 epoxies, 2 polysulphones, platinum, iridium, and titanium. Skin “Patch” tests were applied with all of these ingredients with materials supplied by Medtronic and were negative. When a patch test using a portion of the capsule of a silicone breast implant and silicone gel was applied, the skin test was negative, however, a systemic response manifested by fever, muscle pains, and joint pains ensued which abated immediately following its removal. T cell proliferation response to silica was positive, and IgG and IgM antibodies to silicate were also present. Macrophage derived interleukins, (TNFα, IL-1, IL-6, and IL1-ra), Th1 (IL-2 and IF gamma), and Th2 (IL-4, IL-6 and IL-10) were measured in an effort to identify the relative roles of innate and adaptive specific silica induced immunity in the causation of his illness. The results identified abnormally high levels of all of the macrophage-derived interleukins, normal levels of Th1 and Th2 interleukins, high CD 4/CD 8 ratios, and positive ANAs with topoisomerase and RNP specificities (Lawless, 2001).

Our findings identified that in this patient the defibrillator implant activated all components of the innate and adaptive immune in response to the silicone triggers (and perhaps other of the metals in the implant). Autoantiboides, autoimmunity and an autoimmune disease Sclerodermato-Myositis ensued. Genetic and HLA typing revealed DR2 and DR 3 specificities both of which are associated with auto-immune susceptibility and are seen in Lupus (Grumet and McDevitt, 1972, Lawless et al, 1981), Diabetes (Nerup, 1974, Wucherpfennig, 2001), and Thyroid diseases. Following the use of an anti-TNF treatment for three months (TNF R fusion protein Enbrel/Etanercept) his illness abated, went into complete remission, and did not return. These results confirm our interpretation of the mechanism of his illness-synthetic (silicones) and natural chemicals (silica and silicate and or others) triggered a rare autoimmune illness in a genetically susceptible male.

This finding is further supported by the discovery of incomplete remission of Arthritis and Vasculitis associated with silicone breast implants in a patient following implant removal but complete remission following treatment with monoclonal anti-TNF treatment (Remicade/infliximab). This patient had been treated for over 20 years previously with corticosteroids and methotrexate without success, manifested positive T cell responses in vitro to silica antigens. Following the anti-TNF treatment Remicade/Infliximab, the corticosteroids could be withdrawn, and the patient went into complete remission (Lawless, 2001).

A further patient with a silastic (a solid silicone) wrist implant who developed a severe inflammatory arthritis (rheumatoid like) had a 50% improvement (manifested by reduced pain, swelling, wrist size, and improved motion) within one month following intra-articular injection of 1 mg of Remicade (Lawless, 2001).

Atypical versus Typical Connective Tissue Diseases. The following illnesses are classified as connective tissue diseases or auto-immune diseases, depending on their fulfillment of ACR citeria: Rheumatoid Arthritis (Arnett et al, 1988), Systemic Lupus Erythematosus (Tan et al, 1982), Progressive Systemic Sclerosis or Scleroderma (Seibold, 1994), Polymyositis, Dermatomyositis (Reimer et al, 1986), Mixed Connective Tissue Disease, and Sjogren's Syndrome (Moutsopoulos, 1994). All of the aforementioned diseases are of unknown cause and therefore termed idiopathic in etiology. Both genes and environmental factors are known to play a significant role in their causation and manifestation. Silica exposure and silicosis are known to be risk factors for the development of Progressive Systemic Sclerosis, however, a mechanism has yet to be defined (Rodnan, 1967; Steenland and Brown, 1995; Parks et al, 1999; Cooper et al, 1999; Shero, 1986). Epidemiological evidence suggests an association between different forms of Arthritis in those persons exposed to environmental silica. Several new studies have found evidence of silicone, silica and silicate specific T cell immune responses in Mixed Connective Tissue Disease (Lawless, 2001), (Love 2002) Progressive Systemic Sclerosis (Lawless, 2001), Polymyositis (Miller Lawless, 2001), and Rheumatoid Arthritis associated with Vasculitis (Lawless, 2001). Gold, mercury, lead, vinyl chloride, manganese, iron, nickel, zinc, tungsten, titanium and aluminum have all been reported to be associated with Interstitial Lung Fibrosis in the occupationally exposed with increased levels measured in their lung biopsies as well (Speizer, 2000; Kelleher, 2000). These findings suggest the importance of the route of entry of these chemicals into the body, and that ambient exposures in non-occupationally exposed but genetically susceptible individuals may be sufficient to initiate these connective tissue or auto-immune diseases.

The use of TNFα, IL-1, and IL-6 antagonists provides a unique new tool for the treatment of these illnesses. This applies to both the CIIDs currently thought to be untreatable as well as those which are treatable, but with major negative side effects (by corticosteroids, Methotrexate, Cytoxan, Imuran, Cyclosporine A, or Leflunomide).

Wegener's Granulomatosis. Wegener's Granulomatosis is a systemic illness of unknown causation and uncertain treatment, affecting multiple systems throughout the body including the ear, nose, throat, lungs, skin, kidneys, and eyes. Some symptomatic improvements have been associated with the use of the corticosteroids methotrexate and with cytoxan. The outcomes for survival of greater than 5 years are poor. A new treatment is an urgent necessity. Recently we have identified in vitro silica-specific T cell reactivity in this illness, which in conjunction with epidemiological studies in Europe suggest an environmental exposure to silicone or silica as causative (Lawless and Ojo-Amaize, 1995). Thus, the use of TNF, IL-1, and IL-6 antagonists early in the onset of illness, in combination with removal from the source of exposure provides a promising avenue for the future treatment of this disorder.

Relapsing Polychondritis. Relapsing Polychondritis is a systemic illness involving immune reactivity against extra articular cartilage, such as the ear, the nose, & the trachea. It is of unknown causation and uncertain treatment, affecting multiple systems throughout the body including the ear, nose, throat, lungs, skin, kidneys, and eyes. Some improvements have been associated with the use of the corticosteroids methotrexate and cytoxan. Recently we identified a patient with Relapsing Polychondritis associated with a positive anti-nuclear antibody (Lawless and Ojo-Amaize, 1995). The onset of her disease occurred following silicone breast implantation after mastectomy for breast cancer. Upon removal of the silicone implant there was a significant reduction in the titer of anti-nuclear antibodies and remission of all symptoms associated with the illness. Another patient with this illness in the absence of implants was found to have positive silica specific T cell proliferation responses in vitro (Lawless and Ojo-Amaize, 1995).

Idiopathic and Cryptogenic Non-Infectious Immune and Auto-Immune Lung Diseases. The classical or typical connective tissue diseases, all have a high-frequency of lung involvement in addition to Arthritis. The disease with the highest frequency of Interstitial Lung Fibrosis is Progressive Systemic Sclerosis, followed by Mixed Connective Tissue Disease, Sjogren's syndrome, Systemic Lupus Erythematosus, Polymyositis, and Rheumatoid Arthritis. Rheumatoid Arthritis is termed Caplan's Syndrome when associated with Interstitial Fibrosis in coal workers. All of the aforementioned syndromes and diseases have now been associated with silica exposure, elevated levels of TNF, IL-1, and IL-6 in their plasma, and silica-specific T cell proliferation responses in vitro. As a consequence, a predictable response to the invention is high.

Idiopathic Non-Infectious Disorders of the Brain and Peripheral Nervous System. Alzheimer' Disease (“AD”) and other degenerative dementing illnesses are a major concern for researchers, health economists, health insurances, and congress because of the enhanced life expectancy of the U.S. population. As the fourth leading cause of death in western societies, Alzheimer's disease affects around 15% of those who live to 65, and 35% of those 85 and over (St. George Hyslop 2000). Currently there are an estimated 4 million Americans suffering from these diseases at an estimated cost of care which exceeded $60 billion in 1991 (cite). Alzheimer's disease is characterized by a progressive change in cognition, leading to a progressive memory loss dementia, and personality change eventually requiring total dependent care by a care giver. The disease is characterized radiologically by brain atrophy, pathologically by neuronal cell death from the deposition of the proteins Amyloid (ASP) and Tau, giving rise to amyloid plaques around, and fibrillary tangles Tau, within the cells of the brain-neurons, and their synapses, or nerve ending junctions. There is a genetic component to the disease, and much research has focused on treatment modalities to decrease production of these amyloid and Tau proteins, and or to increase their metabolism, or to block the action of the secretases which have been associated with enzymatically producing the APP form the APP amyloid precursor protein, thereby blocking its formation in the brain tissue. Aluminum, silica, zinc, copper, and iron are all found in brain tissue. Al and Si are found in and around the synapses, which are the sites of cellular communication, and therefore, brain function. One author has coined the term synapatoconiosis, suggesting that this is also a disease caused by chemical environmental exposures similar to the lung in pneumoconiosis. Another has used cephaloconiosis to suggest a similar mechanism (Evans 1991).

Aluminium in drinking water has been cited as a risk factor for both memory loss and AD, and is a known neurotoxin. Recently the latter has been associated with the development of arthritis and macrophagic induced Fasciitis, in individuals vaccinated with Hepatitis B vaccine, where the cause of the illness was found to emanate from Aluminum hydroxide in the vaccine where it was used as an adjuvant. Both aluminum and silicates have been detected in the brains of AD patients and specifically in the cores of the proteins forming these plaques and tangles. Much of the research on this disease has centered on the genetics of the cell producing these amyloid and tau proteins. Much less research has focused on the cause of the inflammation that is evidenced by the findings of the MDIs, TNFα, IL-1, and IL-6 in spinal fluid, in and around the nerve synapses, in brain biopsy and autopsy specimens, and the sites of nerve dropout. Elevated proteins, and oligoclonal bands are also found in the CSF in AD. Both of these findings result from over stimulation of B cells. T cells and B cells are normally not found in the brain. They are found however in AD, and the presence of oligoclonal bands in the CSF is consistent with the concept of persistent innate and specific immune T & B cell stimulation. Monoclonal bands and systemic amyloidosis have been reported in women in association with silicone gel breast implants. A similar mechanism may thus be operational in the brains of AD patients, and in those with renal failure and dialysis induced dementia neuro-myopathy ALS and nephrogenic fibrosing dermatopathy. Because the brain is a privileged site, there should not be present potentially immune-activating metallic chemicals. The brain's protection and nutrition is solely dependent on a normal functioning innate immune system. The presence of CIID chemicals disrupts this normal homeostatic mechanism.

Neural dysfunction is manifested in vivo by decreased neurotransmission of impulses by acetylcholine chemicals at the neural synapses. Microglial cellular activation, proinflammatory cytokine production and deposition, apoptotic neuronal cell death, over-production of amyloid and tau proteins, detection of prostagalandins, reactive oxygen species, peroxynitrite, when all are taken together are in support of the concept that a persistent trigger of MDIs is the cause of neuronal cell death and dysfunction. The suggestions of improvement in memory loss and AD with aspirin, reduction of AD in RA patients, and further improvement in memory with stronger NSAIDs such as Indomethacin, Ibuprofen and Naproxen sodium and perhaps other of the DMARDs used in RA, are in support of this opinion. With the knowledge that silica and silicates causes all of the above findings in vitro and in vivo in the lungs, joints, skin, and tissues of mammals, and that aluminum is used as an ingredient in vaccines for its adjuvant effects in boosting immune responses through enhanced cytokine production at inoculation sites, the evidence is accumulating that persistent innate activation, in an immune privileged site—the brain by these Aluminum and Silica chemical triggers in the brains of AD patients exist, and is likely one mechanism for this disease.

In this regard, AD resembles the mouse model of, and the human disease systemic amyloidosis. In the mouse, persistent innate immune activation by daily immunization with casein (sodium caseinate a milk protein), endotoxin, or LPS, and Silver nitrate (all potent activators of the innate immune system) cause the accumulation of amyloid fibrils in 100% of the liver and spleen of affected mice. The use of anti-inflammatory and immunosuppressive drugs coterminous with the initiation of immunization, blocks disease from developing. These same immunosuppressive drugs accentuate the disease onset and severity when given half way through the immunization process. Human amyloid disease is associated with chronic infectious diseases which are persistent, and even with and silicone (see above), which preferentially and continuously activate the immune system. Examples are Tuberculosis, Leprosy and Staphylocccal Osteomyelitis. We know that the persistent innate stimulation results in the production of MDIs, and is followed by both apoptotic and inflammatory cell death, and that anti-TNF agents result in remission of RA, Crohn's disease, Pyoderma Gangrenosum, silicone/silica/silicate-induced Vasculitis, Arthritis, and Sclerodermatomyositis in a manner far superior to NSAIDs, DMARDs, and other chemotherapeutic drugs with less potential toxicity. We believe that use of these agents at the onset, or as early in AD detection as possible, will impede and prevent the onset and further development of AD with enormous benefits to individuals and society. The method of administration will be necessarily be either intrathecally via lumbar puncture, via cerebro-peritoneal shunt, or intranasal administration. Some researchers have suggested that the portal of entry for the silica and aluminum chemicals is in fact the nasal passages. Predictably, attempts to immunize patients with amyloid AβP had to be abandoned as they caused immune mediated cerebritis.

Peripheral neuropathy is an inflammatory condition of the peripheral nerves found distally throughout the body, which is distinct from illnesses involving the brain. The mechanism however may be the same, and it may coexist with brain cell dysfunction. The causes are multiple: infectious, inflammatory, associated with the connective tissue diseases, vasculitis, amyloidosis, including that with multiple myeloma, degenerative, toxic and metabolic. Of the toxic causes, alcohol and diabetes predominate. Heavy metals such as mercury, lead, aluminum and others have all been cited as causes.

The nerve fiber is composed of an axon, the extended arm of the nerve cell or neuron, which arises from the brain or spinal cord, and is surrounded by a myelin sheath. A third peripheral nerve compartment is called the autonomic nerve system, which is the nerve system responsible for involuntary control of the vital organs within the body (heart, lungs, gut, bladder, bowel, and genital functions as examples) which remain silent unless they are stressed or diseased. Diseases of peripheral nerves as therefore thought to be either axonal, or myelin based. Autonomic nerve dysfunction is rare and is most frequently associated with Amyloidosis and Diabetes. It can however be associated with Scleroderma. When it is present and severe it is associated with profound postural hypotension defined as a drop in systolic pressure of greater than 20 mm of mercury upon standing. We discovered that patients with both silicone breast implants and heart pacemakers developed symptomatic postural hypotension at varying time intervals following their implant procedures, and that several of these were silicone, silica, and silicate T cell reactors to these triggers in vitro. Prompted by these findings we recently studied and reported on three patients who had both postural hypotension, & chronic fatigue, both of which have been previously reported ( ), and also peripheral neuropathy. All three displayed silicone specific T cell reactivity in vitro, in the absence of any form of silicone implants, but with a history of ambient silica exposure. Two of the three were available for, and consented to treatment with anti-TNF biologics. One received Enbrel and the other Remicade. After 3 months of anti-TNF treatments their postural hypotension abated, and they were symptomatically improved. A nerve biopsy of a peripheral nerve in one patient revealed axonal degeneration and attempted re-myelination, or repair. These findings of ambient silica exposure with resultant Postural Hypotension, Chronic Fatigue and both Peripheral and Autonomic Neuropathy, and a response to anti-TNF treatment are consistent with a toxic role for silica exposure on both peripheral and autonomic nerves, via the CIIDs mechanism of MDI over-production, resulting in axonal and myelin degeneration.

Persian Gulf War Illness. About 700,000 U.S. and Allied troops were deployed in the Persian Gulf War. Many returned with an ill-defined illness that has been termed the Persian Gulf War Illness (“PGWI”). Studies of chemical and biological toxins, immunizations and vaccinations, and spent uranium have all been carried out in an effort to understand a mechanism for this illness. None to date have found a cause. Stress has been defined as a significant component, but not the underlying mechanism. What is new and supremely significant is that the symptoms associated with PGWI are similar to those of the SBI in frequency, severity, and duration. Studies on air pollution measurements performed after the combat phase of the war revealed particulate levels of PM 10 size (airborne particles measuring 10 microns in diameter and less), that were between 10-200 times higher than the EPA's acceptable levels for both single and annual individual exposure. The source of the particulates was predominantly wind-blown sand. The sand in the Saudi Arabian desert is fine (PM 1 and less) and remains aerosolized longer after turbulence from wind or vehicular movements. When inhaled as sand, silica in its crystalline, amorphous or (combined with) metallic salt forms can cause pulmonary macrophage activation resulting in the production of the pro-inflammatory interleukins TNF, IL-1, and IL-6. In its crystallized form, silica (silicon dioxide) is difficult to metabolize and eliminate from the human body. It will persist, and in genetically susceptible soldiers, give rise to cytokine production, specific T and B cell immunity, and autoimmunity, resulting in a plethora of nonspecific signs and symptoms. No studies have yet been performed to evaluate the potential for silica specific adaptive immunity in these symptomatic soldiers. No treatments are currently available.

Despite expenditures of >$300 million, and hundreds of studies by DOD, VA, NIH, and the work of Commissions and Oversight Committees, no cause for PGWI has yet been found. Between 30-40% of those deployed (900,000) in the first gulf war with the PGWI are sick or disabled. Initially it was estimated that 14% or approximately 100,000 of Gulf War veterans were affected by a health related multi-symptom illness. Currently this percentage has been increased to between 26-40%. Potentially causative environmental exposures include vaccinations, pesticides, oil fires, prydidostigmine, bromide, smoke and dust inhalation. None has yet been proven. The rate of the Illness was lowest (21%) in those who served previously on board ship. Higher (31%) in those who served on land, in support areas. Highest (42%), in those who entered the land war in Iraq and Kuwait. Duration of deployment on land, increased the rates of illness also.

Symptoms, in decreasing frequency, include Headaches, Joint Pains, Fatigue, cognitive difficulties, Diarrhea, Skin Rashes, Respiratory Symptoms, and Dizziness. Longterm affects identified by the VA administration include a doubling of the rate of Amyotrophic Lateral Sclerosis, and possibly, increased rates of Multiple Sclerosis, and Parkinsonism. Subtle neuro-cognitive dysfinction, neurophysiological testing abnormalities, neurohormonal and neuroimaging deficits were also found.

Atmospheric analysis of air samples taken on the ground and in the air downwind from the oil fires after the combat phase of the war, revealed PM10[?] levels which were far higher than acceptable levels of particulates by EPA standards. These particles were defined microscopically to be silica, and caused by, windblown sand. One has to assume that during the combat phase of this war the PM levels were immeasurably higher. Air sampling data, following the Mt St Helens eruption, revealed the silica-rich plume traveled west to east, encircled the globe, and was identified by sampling on the west coast again one week later. Masks or filters were not used or mandated during this war.

Particulates measuring PM 10 or less ie. 2.5, are considered to be inhaled deeper into the lungs and to be the most inflammatory to lung tissues. Saudi sand is finer than PM10, measuring as low as low as 0.1 and has a different chemical structure than regular sand. The consequence of this is that if disturbed, or aerosolized by wind, or heavy vehicles, these particles will remain in the air for a far longer time than heavier sand, and result therefore in a longer duration of exposure for the troops exposed on the ground. No data was available during the combat phase of this war.

Desert Storm Pneumonitis or El Askhan Disease. This illness was named when troops entered a Saudi Arabian village built for the local bedouins and which had remained uninhabited. Upon entry they found it to be wind swept and inhabited only by pigeons and their droppings. The resulting disease was caused by inhalation of the dust, comprised of sand and pigeon droppings. The resultant pneumonitis was a combination of acute Silicosis and External Inhalant Alveolitis due to inhaled pigeon gamma globulin, IgA class, which is found in and aerosolized from the pigeon stool. Both the sand (silica) and the pigeon IgA are known to activate the Monocyte/Macrophage cell population in humans, and to activate specific immune responses in humans with both T cell and B cell (with IgM IgG and IgE in Pigeon Breeder's Disease) antibodies resulting.

World Trade Center Illness. Firemen, Policemen, and Rescue Workers, at the World Trade Center Collapse of 9/11 were exposed to, and inhaled, dust. General Area (GA) and Personal Breathing Zone (PBZ) dust samples were collected and analyzed by NIOSH, for contaminants including Asbestos and Silica. Of 29 bulk samples analyzed 26/29 (90%) had <1% Asbestos, and 3/29 had 1-3% (by mass). 26/27 samples had crystaline silica. Concentrations by mass ranged from 0-18% (median of 3.2%). (MMWR May 31 2002. v51. no21) Silica and Silicate are known to activate innate and adaptive specific cellular and humoral immunity in vivo and ex vivo, with the production of Interleukins-IL-1, IL-6 and TNFα, T cell proliferation, and silicate antibodies of the IgM and IgG classes. Silica inhalation is responsible for lung fibrosis, (Silicosis), acute and chronic respiratory illness, autoantibodies, certain autoimmune and vasculitic diseases. While no studies to date have been performed, it is likely, and we hypothesize, that silica inhalation is responsible for the illness of respiratory inflammation and bronchial hyper-reactivity identified in symptomatic exposed workers with World Trade Center Cough (N E J Med 347:806-815, Sep. 12 2002).

Methods for Diagnosis and Identification of CIID Illnesses.

Diagnosis of CIID illnesses commences with recognition of the Problem, The Illnesses associated, the Cause, and the Mechanism of these Illnesses. All diseases are both Genetic and Environmental and each component has to be pursued in order to identify both susceptibilities and severities, of the responses of, and in, affected individuals.

Identification of CIID triggers in the Air, Food, Water, Vaccines, Implants, Intravenous Stents, Tubing, Shunts, Dialysis membranes, and Heart Lung by pass machines using chemical analysis by those skilled in the art, and or from the manufacturer's composition manuals. Identification of the CIIDs triggers in the body, in the tissues and organs including hair and nails, and in the body fluids such as blood, urine, cerebrospinal, pleural, and synovial using chemical methods by those skilled in the art.

Identification of innate and specific immune activation in the affected individual by the suspected CIID trigger, using a variety of immune assays to include-TOLL receprtors, MDI generation in vivo and ex vivo in response to the suspected CIID chemical, lymphocyte transformation assays, macrophage migration inhibition assays, individual cytokine and antibody assays, & complement fixation assays, by those skilled in this art.

Identification of the activated cells T, B and antibodies in the body at the sites of disease induced by CIID by microscopy-light, fluorescent, and electron, by those skilled in the art.

Identification of the indicted chemical in the tissue or organs affected by electron spin resonance and laser raman spectroscopy by those skilled in the art. Development of Biomarkers for each identified CIID trigger. Examples are Beryllium Lymphocyte Transformation assay in Berylliosis, Silica Lymphocyte transformation assay in Silicosis and Asbestosis, and Silconosis, and the Melisa memory Lymphocyte transformation assay in those reacting to Lead, Mercury, or Aluminum and other of the metallic chemicals, which are causing illness.

Development of markers for mutagenesis and other cytogenetic markers for individual cancers associated with CIID triggers. Examples are Lung Cancer with Silica, and Mesothelioma with Asbestos. The presence and availability of a cytogenetic marker such as oncogenes, and tumor suppressor gene inactivation, by those skilled in the art, may identify those at risk for cancer development following a CIID exposure.

Development of Methods of Prevention and Treatment of CIID illnesses by methods other than anti interleukin treatments to include inhibiting cell signaling, gene activation, protein formation using genomics and proteomics and microassays by those skilled in the art.

Development of methods of removal or neutralization of the CIIDs triggers by methods such as Plasmapheresis, Immunoadsorbent chromatography, Dialysis, and Chelation therapies individualized to each trigger.

Development of methods of blockade of TNF, IL-1, and IL-6 receptor by suppressing MDI formation by regulatory cytokines such as IL-10, Interferon gamma and others, to include Suppressor/Regulator T cells and DCs.

### INCORPORATION BY REFERENCE

All publications, patents, and pre-grant patent application publications cited in this specification are herein incorporated by reference, and for any and all purposes, as if each individual publication or patent application were specifically and individually indicated to be incorporated by reference. Citation of a reference is specifically not an admission that said reference is prior art against any claim or claims of the present application. In the case of inconsistencies the present disclosure will prevail. More specifically, the entire contents of co-pending application Serial Number (not yet assigned, Attorney Docket Number 22326-00002) is hereby incorporated by reference for all purposes.

### LISTING OF REFERENCE

Abbas, Abul, Lichtman, Andrew, Pober, Jordan. Cellular and Molecular Immunology. 1994. WB Saunders Company Publ.

Abbas, A. K., K. M. Murphy & A. Sher. 1996. Functional diversity of helper T lymphocytes. Nature 383: 787.

Aderem A, Ulevitch R J. Toll-like receptors in the induction of the innate immune response Nature. 406.782-787 2000.

Andrade F, Casciola -Rosen L, Rosen A. Apoptosis in systemic lupus erythematosus. Clinical implications. Rheum Dis Clin North Am. 2000 May; 26 (2):215-27.

Angell M. Do Breast Implants cause Systemic Disease. N. Engl. J. Med 1994;330:1748

Arend William P. The Innate Immune System in Rheumatoid Arthritis. Arthritis and Rheumatism 2001; Vol 44 No. 10 2224-2234.

Arnett F C, Harrington S M, Bloch D A, at al. The American Rheumatism Association 1987 revised criteria for classification of rheumatoid arthritis. Arthritis & Rheum 1988; 31: 315-324.

Bach J F The Effect of Infections on Susceptibility to Autoimmune and Allergic Diseases. NE J Med v 347;911-920 2002.

Bianchi C Asbestos exposure and Alzheimers disease. 1986 Ital J Neurol Sci February 7(1):145-51.

Billingham R E, Brent L, & Medawar P B. Demonstration of specific acquired tolerance: Actively acquired tolerance of foreign cells. 1953. Nature 172;603-606.

Breitner J C, Zandi P P., Do Nonsteroidal Anti-inflammatory Drugs Reduce the Risk of Alzheimers Disease.? New Eng J Med 345.21 1567-1568 2001.

Breitner J C. Inflammatory processes and ant-inflammatory drugs in Alzheimer's Disease: a current appraisal. Neurobiol. Aging 17 789-794 1996.

Burnet F M. The Clonal Selection Theory of Antibody Formation. 1959 Publisher Cambridge University Press London.

Bellanti Joseph. Immunology. 1971WB Saunders Co, London

Bachmann, Martin and Kopf, Manfred. On the role of the innate immunity in autoimmune disease. J Exp Med. 193 (12): 47-50.

Baur X. et al. Systemic Sclerosis in German uranium miners under special consideration of autoantibody subsets and HLA class 11 alleles. Respiration 63(6):368-75 1996.

Benoist, Christophe and Mathis, Diane. Autoimmunity provoked by infection: how good is the case for T cell epitope mimicry? Nature Immunology 2001 2, 797-801.

Bickerstaff, MCM et al. Serum amyloid P component controls chromatin degradation and prevents antinuclear autoimmunity. Nature Medicine. 1999 Vol. 5, No 6.694-7.

Bornstein D, Clinical Manifestations of 100 consecutive women with Silicone Breast Implants, Arthritis.& Rheum. 1993; 36: 5117.

Bowcock, Anne M. and Lovett, Michael. Zeroing in on tolerance. Nature Medicine. 2001 Vol 7 No. 3, 279-281.

Bretscher P. Cohn M. The two-signal hypothesis: A theory of self-non self discrimination: paralysis and induction involve the recognition of one and two determinants on an antigen respectively. 1970 Science 169.1042-1049.

Bretscher P, Cohn M. A theory of self non self discrimination. 1970 Science 1 69:1042-1049.

Bridges A J, Conley C, Wang G, Bums D E, Vasey F B. A Clinical and Immunologic evaluation of women with breast implants and symptoms of rheumatic disease. Ann. Intem. Med 1993;118:929.

Brown S L, Pennello G, Berg W A, Soo M S, Middletonn M S. Silicone gel breast implant rupture extracapsular silicone, and health status in a population of women. J. Rheumatology. 2001 May; 28(5): 996-1003.

Bunn C C, Bernstein R M, Mathews M B. Autoantibodies against alanyl-tRNA synthetase and tRNA Ala coexist and are associated with myositis. 1986. J Exp. Med 163: 1281-1291.

Caciola-Rosen L A, Anhalt G, Rosen A. Autoantigens targeted in systemic lupus erythematosus are clustered in the two populations of surface structures on apoptotic keratinocytes. J. Exp. Med 1994 April 1;179(4):1083.

Casciola-Rosen L, Wigley F, Rosen A. Scleroderma autoantigens are uniquely fragmented by metal catalyzed oxidation reactions: implications for pathogenesis. J. Exp. Med. 1997 January 6; 185(1):71-9.

Candy J M et al Aluminosilicates and senile plaque formation in Alzheimers disease. 1986 Lancet February 15; 1 (8477):354-7.

Casciola-Rosen L, Andrade F, Ulanet D, Wong W B, Rosen A. Cleavage by granzyme B is strongly predictive of autoantigen status: implications for the initiation of autoimmunity. J. Exp. Med 1999 September 20;190(6):815-26.

Castronova V et al Silicosis and Coal Workers Pneumoconiosis Environ Health Persp. Vol. 108 Suppl. 4 p 675-684.

Cella, M., F. Facchetti, A. Lanzavecchia & M. Colonna. 2000. Plasmacytoid dendritic cells activated by influenza virus and CD40L drive a potent TH1 polarization. Nat. Immunol. 1: 305. Chrousos, G. P. 1995. The hypothalamic-pituitary-adrenal axis and immune-mediated inflammation [see comments]. N. Engl. J. Med. 332: 1351-1362. Prevention. National Report on Human Exposure to Environmental Chemicals. March 2001.

Chiller J M, Habicht G, Weigle W O. Cellular sites of immunologica unresponsiveness. 1970. Proc. Natl. Acad. Sci. USA 65; 551-556.

Cocco R E, Ucker D S. Distinct Modes of Macrophage Recognition for Apoptotic and Necrotic Cells are Not Specified Exclusively by Phosphatidylserine Exposure. Mol. Biol Cell April 2001;12 (4):929-930.

Coleman C W Homeostatic Processes in Brain Aging; The role of Apoptosis, Inflammation, and Oxidative Stress in Regulating Healthy Neural Circuitry in the Aging Brain, in The Aging Mind: Appendix B 2000. Nat'l Academy Press p 114, Cytokine Induced Pathology Part A; INTERLEUKINS AND GROWTH FACTORS. VOL 34 & 35 Academic Press 1994.

Corrigall V, O'Neill A J Using Immunotherapy to treat rheumatic diseases Trends in Immunol vol 23 no 8 p384 2002.

Fearon D T, Locksley R M The instructive role of innate immunity in the acquired immune response. 1996 science 272:50-53.

Cuellar J L, Scopelitis E, Citera G. A Prospective clinical evaluation of 300 women with Silicone Breast Implants. Arthritis. & Rheum 1993; 36; 5219.

D'Cruz D, Autoimmune diseases associated with drugs, chemicals and environmental factors. Toxicol Lett 2000 March 15; 112-113:421-432.

Cooper G S, Miller F S, Pandey J P. The role of genetic factors in autoimmune disease: implications for environmental research. Environ Health Perspect 1999 October; 107 Suppl 5:693-700.

Davis, Simon J. and van der Merwe, P. Anton. The structure and ligand interactions of CD2: implications fro T-cell function. Immunology Today. 1996.

Dinarello C A, Interleukin I Rev. Inf. Dis. 6.51.1984.

Evans P et al. Oxidative damage in Alzheimers dementia, and the potential etiopathogenetic role of aluminosilicates, microglia and micronuttrient interactions 1992 SXS 62:178-179.

Evans P et al. Cephaloconiosis: a free radical perspective on the proposed particulate induced etiopathogenesis of Alzheimers dementia and related disorders, 1991 Med Hypotheses Mar. 34(3): 209-219.

Feldman M, Brennan M F, Chantry D, Haworth D, Turner et.al. CYTOKINE ASSAYS; Role in evaluation of the pathogenesis of autoimmunity, Immunol. Rev. 119.105 1988.

Gabriel E S, Melton J L, Woods J E, Fallon W M, Kurland L T. Risk of Connective Tissue Diseases and other Disorders after Breast Implantation N. Eng. J. Med 1994; 330: 1967.

Galluci S, Matzinger P. Danger Signals SOS to the imrnune system. 2001. Curr Opin Immunol. 1;13(1):114-119.

Grahame R, Hypermobility Syndrome. 1994 in Rheumatology Klippel J H, Dieppe P A, Eds. 5.188.1.

Grakowi A Et al The Immunological synapse: A molecular machine controlling T-cell activation. Science 285.221-227 1999.

Gregorini G et al.Association between silica exposure and necrotizing crescentic glomerulonephritis with p-ANCA ans anti-MPO antibodies: a hospital based case control study. Adv Exp Med Biol 440:336-435 1993.

Griem P, Wulferink M, Sachs B, Gonzalez J B, Gleichmann E. Allergic and autoimmune reactions to xenobiotics: how do they arise? Immunol Today 1998 Mar. 19 .3.133-141.

Gershon R K, A disquisition on suppressor T cells. Transplant Rev. 1995;26, 170-185.

Hennekens C H, Lee I M, Cook P R, Hebert E W, Karlson E W, LaMotte F, Manson J, Buring J. Self Reported Breast Implants and Connective Tissue diseases in female health professionals. JAMA1996; 255; 616. Hogan S L, Stately K K, Dooley M A. et al. Silica exposure in anti neutrophil cytoplasmic autoantibody associated glomerulonephritis and lupus nephritis. J. Am. Soc Nephrol 12:134-42 2001.

In't Veld Bas A et al Nosteroidal Antiinflammory Drugs and the risk of Alzheimers Disease. New Eng J Med 345.211515-1521 2001.

Isomaki, P. et al. J. Immunol. 2001 166, 5495-5507.

Janeway C A Jr. The immune system evolved to discriminate infectious nonself from noninfectious self. Immunol Today 1992; 13:11-6.

Janeway C A Jr. Approaching the asymptote? Evolution and revolution in immunology. 1999 Cold Spring Harb Quant Biol. 54:11-13.

Janeway C A Jr. The immune system evolved to discriminate infectious non self from noninfectious self. 1992. Immunol Today 13:11-16.

Jacbson A. Lymphocytes recruited as toxic monitors BioMed Net News. Jul. 12 2002.

Kappler J, Roehm N, Marrack P. T Cell tolerance by clonal elimination in the thymus 1987 Cell. 49.; 273-280.

Gershon R K, Kondo K K. Suppressor or regulatory T cells: infectious immunological tolerance. 1971. Immunology 21.903-914.

Grumet M C, Conkell A, Bodmer J G, Bodmer W, McDevitt H O. The MHC in human disease. Histocompatibility antigens associated with systemic lupus erythenmatosus. A possible genetic predisposition to disease. Kaplan M and Richardson B C Mechanisms of Autoimmunity in Environmentally Induced Diseases p 19-29 in Rheumatic Diseases and the Environment Kaufman L D and Varga J Eds Arnold Publ. 1999.

Kollias G, et al. The Role of TNF/TNFR in Organ Specific and Systemic Autoimmunity: Implications for the Design of Optimized “Anti-TNF” Therapies: in Signal Transduction Pathways in Autoimmunity A. Altman Ed Karger Publ. P 30-50. 2001.

Kretz-Romel A, Rubin R L. Disruption of positive selection of thymocytes causes autoimmunity.2000 Nature Med. 6, 298-305.

Kasakawa R, Tojo T, Miyaawaki S, et al. Preliminary diagnostic criteria for the classification of mixed connective tissue disease. 1987; in Mixed Connective Tissue Disease and Antinuclear Antibodies Kasakawa R, Sharp G C, Eds. Amsterdam, Excerpta Medica p. 41.

Kardestuncer T, Frumkin H. Systemic lupus erythematosus in relation to environmental pollution in an African-American community in North Georgia. Arch Environ Health 1997 March-April;52(2):85-90.

Kelleher P et al Inorganic Dust Pneumonias. The Metal Related Parenchymal Disorders. Environ Health Persp. V108 suppl4 p 685-696.

Kondo T, Cortese I, Markovic-Plese S, Wadinger K P, Carter C, Brown M, Leitman S, Martin R. Dendritic cells signal T cells in the absence of exogenous antigen. Nature Immunol. Online 2001 Sep. 17 DOI: 10.1038/ni1711.

Komata T, Tsuchiya N, Matsushita M, Hagiwara K, Tokunaga K. Assocaition of tumor necrosis factor receptor 2 (TNFR2) polymorphism with susceptibility to systemic lupus erythematosus. Tissue Antigens 1999 June; 53(6) 527-533.

Laing T J, Schotenfeld D, Lacey J V, Gillespie B W, Garabrant D H, Cooper B C, Heeringa S G. Alcser K H, Mayes M D, Potential risk factors for undifferentiated Connective tissue disease among women: implanted medical devices. Am. J. Epidemiol. 2001 Oct. 1; 154(7): 610-617.

Lacey J V, Garabrant D H, Laing T J Gillespie B W, Mayes M D, Cooper B C, Schotenfeld D. Petroleum distillate solvents as risk factors for undifferentiated connective tissue diseases (UCTD). Am J Epidemiol 199 Apr 15; 149(8): 761-70.

Lawless O J, Armstrong J H, Berne B H. ds DNA activates suppressor cell activity of normal peripheral blood lymphocytes. ARA Southeastern Meeting 1979.

Lawless O J, Armstrong J H, Suppression of in vitro lymphocyte responses to SK and mitogens by SLE sera with high DNA binding capacity, and accentuation of suppression by added free DNA. 1979. V11 Congresso Panamericano de Reumatologica Bogota Colombia.

Lawless O J, Armstrong J H, Berne BH, Taylor R P, Hooks J. Inhibition of lymphocyte transformation by SLE sera linked to disease activity and DNA binding capacity. ARA meeting Boston 1981.

Lawless O J, Metz S, Light J A, Armstrong J H, Berne B H, Strong D. Association of HLA DR with antibodies to DNA and a subset of SLE patients with nephritis. 15th international Congress of Rheumatology, Paris France June 1981.

Lawless O J, Wistar R, Immune Function in Pigeon Breeders Disease. Fed. Proc. 1971 30(2);396 Abs.

Lawless O, & Ojo-Amaize E, Specialty Labs. Santa Monica Calif. 1995 Unpublished data.

Lawless O.J, Ojo-Amaize E, Agopian M S, Peter J. Silicone Specific T cell proliferative response in women with breast Implants:Correlation with disease Severity 1995 in Immunology of Silicones Workshop, NIH.

Lawless O J, Peterson L. Monoarticular Erosive Arthritis Following Silastic Wrist Prosthesis: Response to Intraarticular Infliximab. Abstract Submitted American College of Rheumatology Meeting San Francisco November 2001.

Lawless O J. Sclero-Dermatomyositis following Pacemaker and Defibrillator Implants: Innate and Adaptive Immune and Cytokine responses to Silica and Remission following Anti TNF treatment suggest a Trigger (Implants) a Mechanism (Innate Activation) and a Treatment (Anti TNF) for the Illness. Abstract Submitted American College of Rheumatology Meeting San Francisco November 2001.

Lawless O J Polyarticular Arthritis and Vasculitis following Silicone Breast Implants: Silicone Specific Immune Responses and Remission following Infliximab Treatemnt. Abstract Submitted American College of Rheumatology Meeting San Francisco November 2001.

Lawless O J, Ojo-Amaize E. Fibromyalgia and Chronic Fatigue following Silicone Implants is Associated with T Cell Specific Proliferative responses to Silicone gel, Silica, and Silicate.Abstract Submitted American College of Rheumatology Meeting San Francisco November 2001.

Lawless O J Chronic Fatigue and Postural Hypotension: Silica Specific T cell Proliferation in the absence of Silicone Implants: Response to Anti TNF treatment Abstract Submitted American College of Rheumatology Meeting San Francisco November 2001.

Lawless O J Is Silicone Implantation an Experiment with Danger Abstract Submitted American College of Rheumatology Meeting San Francisco November 2001.

Lawless O J Chronic Fatigue Postural Hypotension and Silica Specfic Immune Reactivity in the absence of Silicone Implants:Response to Anti TNF Treatment Abstract Submitted American College of Rheumatology Meeting San Francisco November 2001.

Lawless O J Pyoderma Gangrenosum PG and Rheumatoid Arthritis RA Response to Remicade Abstract Submitted American College of Rheumatology Meeting San Francisco November 2001.

Lawless O J Mixed Connective Tissue Disease and Silica Specific Immune Reactivity. Abstract Submitted American College of Rheumatology Meeting San Francisco November 2001.

Lundberg E L, Nyberg P. New developments in the role of cytokines and chemokines in inflammatory myopathies. Current Opinion in Rheumatology 10:521-529 1998.

Lanzavecchia, A. & F. Sallusto. 2001. Regulation of T cell immunity by dendritic cells. Cell 106: 263.

Mosmann, T. R. & R. L. Coffman. 1989. Th1 and Th2 cells: different patterns of lymphokine secretion lead to different functional properties. Annu. Rev. Immunol. 7: 145.

Mosmann, T. R. & S. Sad. 1996. The expanding universe of T-cell subsets: Th1, Th2 and more. Immunol. Today 17: 138-146.

Moutsopoulos H M, Tzioufas A G. Sjogrens Syndrome 1994 in Rheumatology Klippel J H, Dieppe P A. Eds. 6:27.1.

Mandell B F and Calabrese L Laboratory Testing and the Diagnosis of Selected Rheumatic Diseases p 13 in Rheumatic Diseases and the Environment Kaufman L D and Varga J Eds Arnold Publ. 1999.

Miller F W GENETICS OF Environmentally Associsated Rheumatic Disease p 33-39 in Rheumatic Diseases and the Environment Kaufman L D and Varga J Eds Arnold Publ. 1999.

Maverakis, E., van den Elzen, P., and Sercarz, E. E. Self-reactive T cells and degeneracy of T cell recognition: evolving concepts from sequence homology to shape mimicry and TCR flexibility. Journal of Autoimmunity 2001 16: 201-209.

J. Magarian Blander and Ruslan Medzhitov. Regulation of Phagosome Maturation by Signals from Toll-Like ReceptorsScience 2004 304: 1014-1018.

Medzhitov R, Janeway C A Jr. Innate immunity: the virtues of a nonclonal system of recognition. Cell 1997; 91: 295-8.

Medzhitov R, Janeway C A Jr. Innate immunity. N Engl J Med 2000; 343:338-44.

Maloy, Kevin J. and Powrie, Fiona. Regulatory T cells in the control of immune pathology. Nature Immunology. 2001 2: 816-822.

Martinez F D The coming-of-age of the hygiene hypothesis. Respir. Res 2 129-132. 2001.

Matzinger, Polly. Tolerance, Danger, and the Extended Family. Annu Rev Immnunol 1994, 12: 991-1045.

Polly Matzinger Danger Model: A Renewed Sense of Self Science 296: 301-305 2002.

Matzinger P. Tolerance, Danger and the extended family. Ann Rev Immunol 1994 12:991-1045.

Matzinger P. An Innate sense of danger. Seminars in Immunology 1998 10:399-415.

Mason D, Powrie F. Control of Immune Pathology by regulatory T cells Curr. Opin. Immunol. 1998;10: 645-655.

Mevorach, D. et al. Complement-dependent clearance of apoptotic cells by human macrophages. J.Exp. Med. 1998. 188, 2313-2320.

Mosmann T R, Coffman R L. Establishment of the TH1 and TH2 paradigm:TH! And TH 2 cells: different patterns of lymphokine. 1989. Ann. Rev. Immunol 7. 145-173.

McGeer et al. Anti-inflammatory drugs and Alzheimers Disease. Lancet 337.1426 1990.

Nuyts G D, van Vlem E, de Vos A, et al. Wegeners Granulomatosis is associated With exposure to silicon compounds:a case control study. Nephrol Dial Transplant 10: 1162-65 1995.

Kollias G. et al. The Role of TNF/TNFR in Organ Specific and Systemic Autoimmunity: Implications for the Design of Optimized “Anti-TNF” Therapies: in Signal Transduction Pathways in Autoimmunity A. Altman Ed Karger Publ. P 30-50. 2001.

Nathan, Carl and Muller, William A. Putting the brakes on innate immunity: a regulatory role for CD200? Nature Immunology. 2001 Vol 2, No. 117-19.

Naschitz J E Rosner I, Rozenbaum M, Zuckerman E, Yeshurun D. Rheumatic syndromes: clues to occult neoplasia. Semin Arthritis Rheum 1999 August; 29(1): 43-55.

Nicola N A. Guidebook to Cytokines and their receptors Oxford University Press 1995.

Ojo-Amaize E A, Conte V, Lin H C, Brucker R F, Agopian M S, Peter J B. Silicone specific blood lymphocyte response in women with silicone breast implants.

Clin. Diagn. Lab. Immunol. 66: 94-99. 1994.

Ojo-Amaize E, Conte V, Lin H C, Brucker R, Agopian M,Peter J. Silicone Specific blood lymphocyte response in women with silicone breast implants. Clin. Diagn. Lab. Immunol. 1994;1:689.

Ojo-Amaize E, Lawless O J, Peter J. Elevated levels of Interleukin 1beta and Interleukin 1 receptor antagonist in women with silicone breast implants. Clin. Diag. Lab. Immunol 1996;3:257.

Ojo-Amaize E, Lawless O. J, Peter J. Silicone Specific T cell proliferative response in saline breast implant and non breast implant patients. 1995 in Immunology of Silicones Workshop, NIH,

O'Hanlon T P, Okada S, Love la, Dick G O, Young V L, Miller F N. Immunohistopathology & T Cell receptor gene expression in capsules of silicone implants, in Immunology of Silicones Workshop NIH 1995.

O'Hanlon T P, Lawless O J, Katzin W E, Feng L J, Miller F W. Restricted and Shared Patterns of TCR β-chain Gene Expression in Silicone Breast Implant Capsules and Remote Sites of tissue Inflammation. Journal od Autoimmunity 2000 14, 283-293.

Parks C G, Conrad K, Cooper G S. Occupational exposure to crystalline silica and autoimmune disease. Environ Health Perspect 1999 October; 107 Suppl 5: 793-802.

Parks C S et al Occupational Exposure to Crystalline Silica and Risk of Systemic Lupus Erythematosus Arthritis & Rheum v 46 no7 1840-1850 2002.

Pfleiderer B, Garrido L, MMR 1995;33: 8.

Press R I, Peebles C L, Kamugai Y, Ochas R L, Tan E M. Antinuclear Antibodies in women with Silicone Bresast Implants. 1992; Lancet 340: 1304.

Potter M, Morrison S. Plasmacytoma Development in Mice Injected with silicone Gels. Ibid P 397.

Roberts E. Alzheimers disease may begin in the nose and be caused by aluminosilicates. 1986 Neurobiol Aging November-December; 7(6):561-567.

Rondeau V, etal R E; aluminum in drinking water and cognitive decline in elderly the Paquid cohort. Am. J. Epidemiol. 2001 Aug. 1;154(3) 288-290.

Rondeau V et al Relation between aluminum concentrations in drinking water and Alzheimers Disease:an 8 year follow up study. Am J Epidemiol 2000 Jul. 1;152(1):59-66.

Rosen A, Casciola-Rosen L, Wigley F. Role of Metal catalyzed oxidation reactions in the early pathogenesis of scleroderma. Current Opinion in Rheumatology 9:538-543 1997.

Rook G R, Zumla. Gulf War Syndrome: Is it due to a systemic shift in cytokine balance towards a Th2 profile Lancet 349:1831-33 1997.

Rodnan G, Benedek T, Medsger T, Cammarata R, Ann. Inter. Med. 1967; 66;323.

Robnison O G, Bradley El., Disruption Rate of Silicone Gel Prostheses A report of 300 cases. Scientific Abstracts Plastic Surgery 1995; 2.29.

Rejofo M F, Description & characteristics of different silicones including silica as a filler. In Silicone in Medical Devices, Conference Proceddings FDA Center for Devices and Radiological Health Baltimore Maryland. 1991.

Reimer G, Scheer U, Peters J M, Tan E M. Immunolocalization oand partial characterization of a nuclear autoantigen (PM-Scl) associated with polymyositis/sclerodrma overlap syndromes. 1986 J Immunol 137:3802-3808.

Rosen A, Casciola-Rosen L. Autoantigens as substrates for apoptotic proteases: implications for the pathogenesis of systemic disease. Cell Death Differ 1999 January;6(1):6-12.

Rosen A, Casciola-Rosen L. Clearing the way to mechanisms of auotimmunity. 2001 Nature Med. June 7:6;664-665.

Smith M A et al Oxidative stress in Alzheimers disease.2000 Biochimica et Biophysica Acta (BBA)Molecular Basis of Disease. 1502:139-144.

Sakaguchi, S. Animal models of autoimmunity and their relevance to human diseases. Curr Opin Immunol 2000 12, 684-690.

Sanchez-Guerrero J, Schur P H, Sergent J S, Liang M W. Silicone breast implants and rheumatic disease. N E J Med Seibold J R, Systemic Sclerosis in RHEUMATOLOGY, 1994; Klippel J H, Dieppe P A, Eds 6.8.1.

Shen G Q, Ojo-Amaize E, Agopian M S, Peter J. Silicate Antibodies in women with breast implants; Development of a specific assay for detection of humoral immunity. Clin Diag. Lab Immunol 1996: 3: 162.

Saltini D K, Winestock K, Kirby M, Pinkston P, Crystal R G. Maintenance of alveolitis in patients with chronic beryllium disease by beryllium specific helper T cells. N. Eng. J. Med. 1989;320: 1103-1109.

Silverrman S, Vescio R,Silver D, Renner S, Weiner S, Berenson. Silicone gel Implants and Monoclonal Gammopathies: Three case of Multiple Myeloma and Monoclonal Gammopathy of Undetermined Significance. 1996 Current Topics in Microbiology and Immunology 210, Immunology of Silicones. Potter M, Rose N R, Eds page 367.

Sakabe K, Yoshida T, Furuya H, Kayama F, Chan E K. Estrogenic xenobiotics increase expression of SS-A/Ro autoantigens in cultures human epidermal cells. Acta Derm Venerol 1998 November; 78(6): 420-423.

Scott R S, McMahon E J, Pop S M, Reap E A, Caricchio R, Cohen P, Earp S, & Matsushima G K. Phagocytosis and clearance of apoptotic cells is mediated by MER. Nature 2001 411,207-211.

Sharp G C, Irwin W, Tan E M et al Mixed Connective Tissue Disease—an apparently distinct rheumatic disease syndrome associated with a specific antibody to an extractable nuclear antigen(ENA)1977 Am. J. Med 52:148-159.

Shero J H, Bordwell B, Rothfield N F, Eamshaw W C. Autoantibodies to topoismerse 1 are found in sera from scleroderma patients. 1986. Science 231:737-740.

Speizer F E Occupational and Environmental Lung Diseases: An Overview 2000 Environ Health Persp. V108 suppl4 p 604.

Shevach E M,. Regulatory T cella in autoimmunity. Ann. Rev. Immunol 2000;18: 423-449.

Solomon G A, A Clinical and Laboratory profile of symptomatic women with silicone breast implants. Sem. Arthritis. Rheum. 1994 24:29.

Steenland K, Goldsmith D F, Silica Exposure and Autoimmune Disease. Am. J. Ind. Med. 1995; 28: 603.

Steenland K, Brown D, Mortality Study of Gold Miners exposed to Silica and non Asbestiform amphibole minerals. Am. J. Ind. Med. 1995.27;217.

Tan E. Antibody Markers in Systemic Autoimmunity. Diagnostic Studies in Rheumatology Ciba Geigy Publisher Kenneth D. Brandt Ed. 1992.

Tan E M et al Antinuclear antigens (ANAs): Diagnostic and specific immune markers and clues towards the understanding of autoimmunity. 1988. Clin. Immunol. Immunopathol 47: 121-141.

Tan E M, Cohen A S, Fries J F et al The 1982 revised criteria for the classification of systemic lupus erythematosus Arthritis & Rheum. 1982; 25:1272-1277.

Taneja V, David C S. Lessons from animal models for human autoimmune diseases. Nature Immunology 2001; 2, 781-784.

Tervaert J W C, Stegman C A, Kallenberg C G M. Silicon Exposure and vasculitis. Current Opinion in Rheumatology 10:12-17 1998.

Umetsu D et al Asthma: an epidemic of dysregulated immunity. Nat Immunol vol 3 no 8. 715-720. 2002.

Van Dyke K The possible role of peroxynitrite in Alzheimers Disease :a simple hypothesis that could be tested more thoroughly 1997 Med Hypotheses May; 48(5):375-80.

Voll R E Immunosuppressive effects of apoptotic cells. 1997. Nature 390, 350-351.

Watts R A, Scott D G I,and Lane S E Epidemiology of Wegeners Granulomatosis Microscopic Polyangiitis and Churg and Strauss Syndrome p 84 Cleveland Clinic J of Med. Supplement 2, vol 69. 2002.

Weigle, W. O. Analysis of autoimmunity through experimental models of thyroiditis and allergic encephalomyelitis. Adv Immunol 1980 30:159-273.

Wucherpfennig, K. W. Structural basis of molecular mimicry. Journal of Autoimmunity 2001 16: 293-302.

Yashiro M, Muse E, Itch-Ihara T, et al. Significantly high mop-ANCA-relate angiitis and/or nephritis with respiratory tract involvement after the 1995 great earthquake in Kobe Japan Am. J Kid Dis. 35: 889-895. 2000.

Zhang Q et.al. Changes in Immune Parameters Seen in Gulf war Veterans but not in Civilians with Chronic Fatigue syndrome. Clin. Diagn. Lab. Immunol. 6: 6-1 1999.

Shirikawa T, Enomoto T, Shimazu S, Hopkin J M. The inverse association between tuberculin responses and atopic disorder. Science 275.77-79. 1997.

Kiechl S et al Toll-like Receptor 4 Polymorphisms and Atherogenesis N E J Med 347:185-192 2002.

Rehli R Of mice and men: species variations of Toll-like receptor expression TRENDS in Immunology v23 no 8 735-378.2002

Rogge, L., L. Barberis-Maino, M. Biffi, et al. 1997. Selective expression of an interleukin-12receptor component by human T helper 1 cells. J. Exp. Med. 185(5): 825.

Sallusto, F., C. R. Mackay & A. Lanzavecchia. 2000. The role of chemokine receptors in primary, effector, and memory immune responses. Annu. Rev. Immunol. 18: 593.

Shapiro S D, Owen C A ADAM-33 Surfaces as an Asthma Gene NE J Med v 347;936-938. 2002.

Braun-Fahrlander C et al Environmental Exposure to Endotoxin and Its Relation to Asthma in School Children NE J Med v 347;869-877. 2002.

Trinchieri, G. 2003. Interleukin-12 and the regulation of innate resistance and adaptive immunity. Nat. Rev. Immunol. 3: 133-146.

Varga J Scleroderma and Smads: Dysfunctional Smad family dynamics culminating in fibrosis. Arthritis & Rheum v 46 no 7 1703-1713 2002.

Weiss S Eat Dirt—The Hygiene Hypothesis and Allergic Diseases. NE J Med v 347;930-931. 2002

Dekai Zhang, Guolong Zhang, Matthew S. Hayden, Matthew B. Greenblatt, Crystal Bussey, Richard A. Flavell, and Sankar Ghosh Toll-like Receptor That Prevents Infectionby Uropathogenic bacteria Science 2004 303: 1522-1526.

Florian Heil, Hiroaki Hemmi, Hubertus Hochrein, Franziska Ampenberger, Carsten Kirschning, Shizuo Akira, Grayson Lipford, Hermann Wagner, and Stefan Bauer Species-Specific Recognition of Single-Stranded RNA via Toll-like Receptor 7 and 8 Science 2004 303: 1526-1529.

Recent References on the Nature of TOLL Receptors.

Sandra S. Diebold, Tsuneyasu Kaisho, Hiroaki Hemmi, Shizuo Akira, and Caetano Reise Sousa Innate Antiviral Responses by Means of TLR7-Mediated Recognition of Single-Stranded RNA Science 2004 303: 1529-1531.

Luke A Oneill After the TOLL Rush, Science, Vol 303, Issue 5663,1481-1482 March 2004.

Robin D. Hatton and Casey T. Weaver T-bet or Not T-bet Science 302: 993-994.

Gregory M. Barton and Ruslan Medzhitov Toll-Like Receptor Signaling Pathways Science 300: 1524-1525.

Chandrashekhar Pasare and Ruslan Medzhitov Toll Pathway-Dependent Blockade of CD4+CD25+T Cell-Mediated Suppression by Dendritic Cells Science 299: 1033-1036. Published online Jan. 16, 2003; 10.1 126/science.1078231 (Science Express Research Articles).

Shohei Hori, Takashi Nomura, and Shimon Sakaguchi Control of Regulatory T Cell Development by the Transcription Factor Foxp3 Science 299: 1057-1061. Published online Jan. 9, 2003; 0.1126/science. 1079490 (Science Express Reports).

Carl Nathan Catalytic Antibody Bridges Innate and Adaptive Immunity Science 298: 2143-2144.

Mark S. Anderson, Emily S. Venanzi, Ludger Klein, Zhibin Chen, Stuart P. Berzins, Shannon J. Turley, Harald von Boehmer, Roderick Bronson, Andree Dierich, Christophe Benoist, and Diane Mathis Projection of an Immunological Self Shadow Within the Thymus by the Aire Protein Science 298: 1395-1401. Published online Oct. 10, 2002; 10.1126/science. 1075958 (Science Express Research Articles).

Andrew L. Singer and Gary A. Koretzky Control of T Cell Function by Positive and Negative Regulators Science 296: 1639-1640.

Stephen B. Gauld, Joseph M. Dal Porto, and John C. Cambier B Cell Antigen Receptor Signaling: Roles in Cell Development and Disease Science 296: 1641-1642.

Eliot Marshall. Lupus: Mysterious Disease Holds It's Secrets Tight Science 296: 689-691 2002.

Maria Yazdanbakhsh, Peter G. Kremsner, and Ronald van Ree Allergy, Parasites, and the Hygiene Hypothesis Science 296: 490-494 2002.

Ruslan Medzhitov and Charles A. Janeway, Jr. Decoding the Patterns of Self and Nonself by the Innate Immune System Science 296: 298-300 2002.

P. Anton van der Merwe and Simon J. Davis The Immunological Synapse—a Multitasking System Science 295: 1479-1480 2002.

Kyeong-Hee Lee, Amy D. Holdorf, Michael L. Dustin, Andrew C. Chan, Paul M. Allen, and Andrey S. Shaw T Cell Receptor Signaling Precedes Immunological Synapse Formation Science 295: 1539-1542. 2002.

Having thus described our invention, what we claim as new, and desire to secure by Letters Patent is:

1. A pharmacological composition for treating chemically-induced immune disorders in a mammal comprising:
at least one TNF antagonist;
at least one IL-1 antagonist; and
at least one IL6R antagonist.

2. The pharmacological composition for treating chemically-induced immune disorders in a mammal, according to claim 1, wherein said at least one TNF antagonist is selected from the group consisting of etanercept, infliximab, and mixtures thereof.

3. The pharmacological composition for treating chemically-induced immune disorders in a mammal, according to claim 1, wherein said at least one IL-1 antagonist is selected from the group consisting of kineret.

4. The pharmacological composition for treating chemically-induced immune disorders in a mammal, according to claim 1, wherein said at least one IL6R antagonist is selected from the group consisting of MRA, and D2EF Adalimubab, and mixtures thereof.

5. The pharmacological composition for treating chemically-induced immune disorders in a mammal, according to claim 1, further comprising at least one corticosteroid.

6. The pharmacological composition for treating chemically-induced immune disorders in a mammal, according to claim 5, wherein said corticosteroid is selected from the group consisting of methotrexate, leflunomide, cytoxan, immuran, cyclosporin A, and mixtures thereof.

7. A method of treating chemically-induced immune disorders in a mammal comprising administering to said mammal a pharmacological composition comprising:
at least one TNF antagonist;
at least one IL-1 antagonist; and
at least one IL6R antagonist.

8. The method of treating chemically-induced immune disorders in a mammal, according to claim 7, wherein said at least one TNF antagonist is selected from the group consisting of etanercept, infliximab, and mixtures thereof.

9. The method of treating chemically-induced immune disorders in a mammal, according to claim 7, wherein said at least one IL-1 antagonist is selected from the group consisting of kineret.

10. The method of treating chemically-induced immune disorders in a mammal, according to claim 7, wherein said at least one IL6R antagonist is selected from the group consisting of MRA, and D2EF Adalimubab, and mixtures thereof.

11. The method of treating chemically-induced immune disorders in a mammal, according to claim 7, wherein said pharmacological composition further comprises at least one corticosteroid.

12. The method of treating chemically-induced immune disorders in a mammal, according to claim 11, wherein said corticosteroid is selected from the group consisting of methotrexate, leflunomide, cytoxan, immuran, cyclosporin A, and mixtures thereof.
